(19) United States
(12) Patent Application Publication
(10) Pub. No.: US 2025/0325598 A1
(43) Pub. Date: Oct. 23, 2025

(54) IMMUNOBIOTICS FOR PREVENTING BACTERIAL PNEUMONIA AND METHODS OF USING THE SAME

(71) Applicant: The Regents of the University of Colorado, A Body Corporate, Denver, CO (US)

(72) Inventor: Sarah E. Clark, Denver, CO (US)

(21) Appl. No.: 18/863,566

(22) PCT Filed: May 8, 2023
(86) PCT No.: PCT/US2023/066719

§ 371 Date: Nov. 6, 2024

Related U.S. Application Data
(60) Provisional application No. 63/339,215, filed on May 6, 2022.

(51) Int. Cl. A61K 35/74; A61K 39/00; A61K 39/02; A61P 31/04; A61P 37/04

(52) U.S. Cl. CPC A61K 35/74; A61K 39/0208; A61P 31/04; A61P 37/04; A61K 2039/521

(57) ABSTRACT

Methods for treating or preventing bacterial pneumonia in a subject involve administration of a Prevotella composition to the subject. The Prevotella composition may include cells, or portions thereof, of one or more of P. melaninogenica, P. buccae, P. tannerae, and P. nanceiensis. The composition may be administered by inhalation.

### CROSS REFERENCE TO RELATED APPLICATION

The present application claims the benefit of priority under 35 U.S.C. § 119 to U.S. Provisional Patent Application No. 63/339,215, entitled “AIRWAY IMMUNOBIOTICS FOR PNEUMOCOCCAL PNEUMONIA,” filed May 6, 2022, the entirety of which is hereby incorporated by reference herein for all purposes.

### STATEMENT OF GOVERNMENT INTEREST

This invention was made with government support from the National Institute of Allergy and Infectious Disease of the National Institutes of Health under grant number K22AI143922. The government has certain rights in the invention.

### TECHNICAL FIELD

The present disclosure relates generally to compositions and methods for treating or preventing a respiratory infection. Specific implementations include administration of a Prevotella immunobiotic to enhance protection against bacterial pneumonia.

### BACKGROUND

Streptococcus pneumoniae is the most common cause of community-acquired pneumonia, which has an estimated economic impact of $17 billion annually in the U.S. alone and is the leading cause of death in children under five years old. Staphylococcus aureus is the most common cause of hospital-acquired pneumonia. Pneumococcal vaccines are generally effective against invasive disease, but are not as protective against pneumonia. Treatment of S. pneumoniae and/or S. aureus infections is complicated by the rising prevalence of antibiotic-resistant strains and non-vaccine serotypes. Accordingly, compositions and methods for treating or preventing bacterial pneumonia are needed.

### SUMMARY

Embodiments disclosed herein generally relate to methods for treating or preventing bacterial pneumonia in a subject by pulmonary administration of a Prevotella composition.

In accordance with embodiments of the present disclosure, a method of treating bacterial pneumonia may involve administering a therapeutically effective amount of a composition comprising cells, or portions thereof, of one or more strains of Prevotella. In accordance with embodiments of the present disclosure, a method of promoting clearance of pneumonia-causing bacteria from a lung may involve administering a composition comprising cells, or portions thereof, of one or more strains of Prevotella. The pneumonia-causing bacteria may be one or more of Streptococcus pneumoniae and Staphyloccocus aureus. In accordance with embodiments of the present disclosure, a method of reducing one or more symptoms caused by bacterial pneumonia may involve administering a therapeutically effective amount of a composition comprising cells, or portions thereof, of one or more strains of Prevotella. In some examples, the one or more strains of Prevotella comprise P. melaninogenica, P. buccae, P. tannerae, or P. nanceiensis. In some examples, the composition is administered by inhalation.

In accordance with embodiments of the present disclosure, an inhalable immunobiotic composition may include cells, or portions thereof, of one or more strains of Prevotella. In some examples, the one or more strains of Prevotella include P. melaninogenica, P. buccae, P. tannerae, or P. nanceiensis. In some examples, the portions of cells include lipoproteins. In some examples, the cells of at least one of the one or more strains of Prevotella are live. The live cells may be present at about 10⁵ to about 10⁷ CFU. In some example, the cells, or portions thereof, of at least one of the one or more strains of Prevotella are inactivated. The inactivated cells, or portions thereof, may present at about 10⁷ CFU equivalents.

In accordance with embodiments of the present disclosure, a method of promoting neutrophil activation in a lung may involve administering a composition comprising cells, or portions thereof, of one or more strains of Prevotella. In some examples, the one or more strains of Prevotella include P. melaninogenica, P. buccae, P. tannerae, or P. nanceiensis. In some examples, the composition is administered by inhalation.

In accordance with embodiments of the present disclosure, a method of activating, enhancing, and/or promoting an innate immune response in a subject afflicted with a respiratory infection may involve administering a composition comprising cells, or portions thereof, of one or more strains of Prevotella. In some examples the method includes increasing a presence of neutrophils in one or both lungs of the subject. Increasing the presence of neutrophils may cause a lung-localized increase in TNFα production followed by IL-10 production. In some examples, the method includes sub-clinical inflammation followed by inflammatory resolution.

This Summary is neither intended to be, nor should it be, construed as being representative of the full extent and scope of the present disclosure. Moreover, references made herein to “the present disclosure,” or aspects thereof, should be understood to mean certain embodiments of the present disclosure and should not be construed as limiting all embodiments to a particular description. The present disclosure is set forth in various levels of detail in this Summary as well as in the attached drawings and Detailed Description and no limitation as to the scope of the present disclosure is intended by either the inclusion or non-inclusion of elements, components, etc. in this Summary. Features from any of the disclosed embodiments may be used in combination with one another without limitation. In addition, other features and advantages of the present disclosure will become apparent to those of ordinary skill in the art through consideration of the following Detailed Description and the accompanying drawings.

### BRIEF DESCRIPTION OF THE DRAWINGS

The drawings illustrate several embodiments of the disclosure, wherein identical reference numerals refer to identical or similar elements or features in different views or embodiments shown in the drawings.

FIG. 1 is a graph showing the levels, in colony forming units (CFU), of S. pneumoniae detected in the lungs of mice exposed intratracheally (“i.t.”) to various indicated doses of live P. melaninogenica for 24 hours prior to a 24-hour S. pneumoniae infection.

FIG. 2 is a graph showing the survival of mice following exposure to heat-killed (“HK”) P. melaninogenica for 24 hours prior to a 24-hour S. pneumoniae infection.

FIG. 3 is a graph showing the level, in CFU, of S. pneumoniae detected in the lungs of mice exposed i.t. to P. melaninogenica HK, E. coli HK, or E. coli lipopolysaccharide (“LPS”) for 24 hours prior to a 24-hour S. pneumoniae infection.

FIG. 4 is bar graphs showing the levels of serum TNFα or IL-10 in mice exposed i.t. to P. melaninogenica HK, E. coli HK, or E. coli LPS for 24 hours prior to a 24-hour S. pneumoniae infection.

FIGS. 5A-5E are graphs showing the effects of P. melaninogenica HK on the innate immune system of mice in the absence of S. pneumoniae infection. Levels of cytokines and chemokines in bronchoalveolar lavage fluid (FIG. 5A), TNFα and IL-10 in serum (FIG. 5B), inflammatory monocytes (FIG. 5C), neutrophils (FIG. 5D), and TNFα in lung neutrophils (FIG. 5E) are shown.

FIGS. 6A-6E are graphs showing the impact of neutrophil or TNFα depletion on P. melaninogenica-mediated protection from S. pneumoniae in mice. FIG. 6A shows intracellular flow cytometry results (left side) and quantification of the same (right side) from infected mice with or without neutrophil depletion. Also shown are the levels of S. pneumoniae detected in the lungs of mice exposed i.t. to P. melaninogenica HK for 24 hours prior to a 24-hour S. pneumoniae infection, with or without neutrophil depletion (FIG. 6B) or TNFα depletion (FIG.

6E). For TNFα depletion, levels of neutrophils (FIG. 6C) and neutrophil TNFα production (FIG. 6D) are also shown.

The CFU levels of S. pneumoniae detected in the lungs of antibiotic treated (microbiome depleted) or Germ-free mice exposed i.t. to live P. melaninogenica for 24 hours prior to a 24-hour S. pneumoniae infection are shown in FIGS. 7A and 7B, respectively. Levels of neutrophils and neutrophil TNFα production from Germ-free mice are shown in FIG. 7C.

FIGS. 8A, 8B, and 8D are graphs showing supernatant TNFα levels 24 hours following incubation of bone marrow neutrophils from naive mice with P. melaninogenica HK (FIG. 8A), with and without inhibitors of TLR2 (C29) or TLR4 (TAK-242) (FIG. 8B), or with and without lipase treatment (FIG. 8D). FIG. 8C is a graph showing shows supernatant TNFα levels 24 hours following incubation of bone marrow neutrophils from naive or Tlr2−/− mice with P. melaninogenica HK, P. melaninogenica HK LPS, P. melaninogenica HK lipoproteins, or the TLR2 agonist Pam3SK4. FIGS. 8E and 8F show CFU levels of S. pneumoniae detected in the lungs of mice exposed to P. melaninogenica HK or lipoprotein-digested P. melaninogenica HK (FIG. 8E), or P. melaninogenica lipoproteins or Pam3SK4 (FIG. 8F) i.t. prior to a 24-hour S. pneumoniae infection.

FIGS. 9A and 9B show the percentage of neutrophils (FIG. 9A) or representative flow cytometry plots and total cell numbers of neutrophil TNFα (FIG. 9B) detected by intracellular flow cytometry in wild-type or Tlr2−/− mice treated with P. melaninogenica HK i.t. for 24 hours without infection by S. pneumoniae. FIGS. 9C-9F are graphs showing lung type 2 S. pneumoniae burdens (FIG. 9C), percentage of neutrophils (FIG. 9D), percentage of inflammatory monocytes (FIG. 9E), and percentage of neutrophil TNFα (FIG. 9F) detected by intracellular flow cytometry in mice treated with P. melaninogenica HK i.t. prior to a 24-hour S. pneumoniae infection.

FIGS. 10A-10G show various aspects of lung neutrophil killing of S. pneumoniae. FIG. 10A is a schematic of lung neutrophil purification. FIG. 10B is a bar graph showing the percent of type 2 S. pneumoniae killed by lung neutrophils purified from mice exposed to either E. coli LPS or P. melaninogenica HK i.t. for 24 hours following a 1-hour incubation with opsonized S. pneumoniae. FIG. 10C is a bar graph showing area under curve for total reactive oxygen species (ROS) produced in 1 hour by lung neutrophils purified from mice exposed to either E. coli LPS or P. melaninogenica HK i.t. for 24 hours. FIG. 10D is a bar graph showing serine protease activity for cathepsin G and elastase with or without a protease inhibitor cocktail detected by substrate cleavage for lung neutrophils purified from wild-type (WT) mice exposed to either E. coli LPS or P. melaninogenica HK. FIG. 10E shows the percent of S. pneumoniae killed by lung neutrophils purified from WT mice exposed to P. melaninogenica HK i.t. for 24hours following a 1-hour incubation with opsonized S. pneumoniae in the presence of the ROS inhibitor DPI, protease inhibitors, or no inhibitors. FIGS. 10F and 10G are bar graphs showing the percent of S. pneumoniae killed by lung neutrophils purified from WT or Tlr2−/− mice exposed to P. melaninogenica HK i.t. for 24 hours (FIG. 10F) or serine protease activity with or without protease inhibitor cocktail (FIG. 10G).

FIGS. 11A-11F show the involvement of IL-10 in P. melaninogenica-mediated protection against a 24-hour type 2 S. pneumoniae infection. FIG. 11A is a graph showing levels of cytokines and chemokines in bronchoalveolar lavage fluid following exposure to P. melaninogenica HK in mice infected with S. pneumoniae. FIGS. 11B and 11C are graphs showing lung S. pneumoniae burdens (FIG. 11B) and serum TNFα (FIG. 11C) detected in WT or Il10−/− mice exposed to P. melaninogenica HK. FIGS. 11D-11F show representative flow cytometry plots and total cell numbers of neutrophil TNFα (FIG. 11D), percentage of inflammatory monocyte TNFα (FIG. 11E), and percentage of AM TNFα (FIG. 11F) detected by intracellular flow cytometry from WT or Il10−/− mice treated with P. melaninogenica HK.

FIG. 12A is a graph showing the CFU levels of S. pneumoniae detected in the lungs of mice exposed i.t. to various live Prevotella strains for 24 hours prior to a 24-hour S. pneumoniae infection. FIG. 12B is graphs showing supernatant cytokines TNFα and IL-10 detected 24 hours following incubation of BM neutrophils with various heat-killed Prevotella strains.

FIG. 13A is a graph showing the level of S. pneumoniae detected in the lungs of mice exposed intratracheally to P. melaninogenica HK for 24 hours prior to a 24-hour S. pneumoniae infection. FIG. 13B is a bar graph showing the percent of S. pneumoniae killed by lung neutrophils purified from mice exposed to either E. coli LPS or P. melaninogenica HK i.t. for 24 hours following a 1-hour incubation with opsonized S. aureus.

### DETAILED DESCRIPTION

The present disclosure relates generally to compositions and methods for treating, preventing, reducing the likelihood of contracting, and/or alleviating at least one symptom of a respiratory infection. Specific implementations involve the administration of an immunobiotic composition to the airway of a subject afflicted with, or at risk of developing, bacterial pneumonia. In some implementations, the immunobiotic composition creates or restores a healthy airway microbiome. In some examples, the immunobiotic composition includes cells, or portions thereof, of one or more species of Prevotella, including, but not limited to, P. melaninogenica, P. buccae, P. tannerae, and/or P. nanceiensis.

An immunobiotic composition disclosed herein may be administered one or more times before and/or after a subject contracts or is diagnosed with a respiratory infection, including bacterial pneumonia. Administration of the immunobiotic composition in the manner disclosed, e.g., via inhalation, may increase the levels of Prevotella present in the respiratory tract of a subject, including the mouth, nose, throat, and/or one or both lung(s), where the bacteria may enhance the protection against one or more bacterial species that are capable of causing bacterial pneumonia. Non-limiting examples include species of Streptococcus, including Streptococcus pneumoniae, and/or species of Staphylococcus, including Staphylococcus aureus. Administration of the immunobiotic composition may activate or enhance the innate immune response to infection within the respiratory tract, for example by improving immune cell-mediated clearance of bacterial pathogens from the lung and reducing infection-associated lung inflammation. As a result, one or more symptoms indicative of a respiratory condition, non-limiting examples of which may include coughing, difficulty breathing, sore throat, and/or fever, may be reduced or eliminated. These benefits may prevent, ameliorate, and/or impede the short-and long-term effects associated with respiratory infections in a safe, effective manner.

Without being limited to any mechanism or mode of action, lipoproteins present on the surface of cells of one or more species of Prevotella, including P. melaninogenica, may contribute to protection against one or more species of Streptococcus, including Streptococcus pneumoniae, and/or one or more species of Staphylococcus, including Staphylococcus aureus. The protective lipoproteins may additionally or alternatively be excreted from the cells of one or more species of Prevotella. The lipoproteins may be recognized by toll-like receptor (TLR) 2, and may induce TNFα secretion and neutrophil recruitment in the respiratory tract of a subject administered an immunobiotic composition disclosed herein.

As used herein, “subject” means a human or other mammal. Non-human subjects may include, but are not limited to, various mammals such as domestic pets and/or livestock. A subject may be considered in need of treatment. The disclosed compositions and methods may be effective to treat healthy human subjects, patients diagnosed with a respiratory condition, or patients experiencing one or more symptoms of a respiratory condition.

Treating a respiratory infection, as contemplated herein, encompasses treating, reducing the risk of, preventing, or alleviating at least one symptom of a respiratory infection, which may be caused by the presence or proliferation of one or more species of Streptococcus and/or Staphylococcus in the respiratory tract of a subject. Accordingly, “treating,” “alleviating,” or “preventing,” or any variation thereof, refers to both therapeutic treatment and prophylactic measures, wherein the object is to reduce the likelihood of or slow down (lessen) the targeted pathological condition and/or symptom. Those in need of “treatment” include those already diagnosed with the condition, as well as those prone to contracting or developing the condition. A subject is successfully “treated” if, after receiving a therapeutically effective amount of a pharmaceutical composition according to methods of this disclosure, the subject shows observable and/or measurable reduction in, or absence of, one or more of coughing, fever, chills, fatigue, difficulty breathing, and/or mucus build-up in the respiratory tract. Treating may also encompass enhanced protection against S. pneumoniae or S. aureus, which may encompass or be associated with clearance of S. pneumoniae or S. aureus, respectively, from the lung(s) of a subject.

“Reducing,” “reduce,” or “reduction” means decreasing the severity, scope, frequency, or length of a respiratory condition and/or one or more symptoms thereof.

An “effective amount” of an immunobiotic composition containing cells, or portions thereof, of one or more species of Prevotella is an amount sufficient to carry out a specifically stated purpose, and may be determined empirically and in a routine manner, in relation to the stated purpose. For example, an “effective amount” as used herein may be defined as an amount of an immunobiotic composition that, upon administration to a subject, will reduce the level of one or more bacteria, such as one or more species of Streptococcus and/or Staphylococcus, in the respiratory tract of the subject. The term “therapeutically effective amount” refers to an amount of an immunobiotic composition containing cells, or portions thereof, of one or more species of Prevotella that will treat, reduce the risk of, prevent, or alleviate at least one symptom of a respiratory condition in a subject.

“Administration of” and “administering a” compound, composition, or agent should be understood to mean providing a compound, composition, or agent, a prodrug of a compound, composition, or agent, or a pharmaceutical composition as described herein. The compound, agent, or composition may be provided or administered by another person to the subject or it may be self-administered by the subject, for example using an inhaler or intranasal administration device.

“Pharmaceutical compositions” or “pharmaceutical formulations” are compositions that include an amount (for example, a unit dosage) of one or more of the disclosed Prevotella cells, or portions thereof, together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

As used herein, a “pharmaceutically acceptable excipient” or a “pharmaceutically acceptable carrier” means a pharmaceutically acceptable material, composition, or vehicle involved in giving form or consistency to the pharmaceutical composition. Each excipient or carrier should be compatible with other ingredients of the pharmaceutical composition when comingled such that interactions that would substantially reduce the efficacy of the Prevotellaformulations of this disclosure when administered to a subject and interactions that would result in pharmaceutical compositions that are not pharmaceutically acceptable are avoided. In addition, each excipient or carrier should be of sufficiently high purity to render it pharmaceutically acceptable.

The singular terms “a,” “an,” and “the” include plural referents unless context clearly indicates otherwise. Similarly, the word “or” is intended to include “and” unless the context clearly indicates otherwise. The term “comprises” means “includes.” Also, “comprising A or B” means including A or B, or A and B, unless the context clearly indicates otherwise. The term “about” intended to include values or amounts up to and including 10% greater than or less than the recited value or amount. It is to be further understood that all molecular weight or molecular mass values given for compounds are approximate, and are provided for description. Although methods and materials similar or equivalent to those described herein can be used in the practice or testing of this disclosure, suitable methods and materials are described below. In addition, the materials, methods, and examples are illustrative only and not intended to be limiting.

Unless otherwise defined, all technical and scientific terms used herein have the same meaning as commonly understood by one of ordinary skill in the art to which this disclosure belongs. In the case of conflict, the present specification, including definitions, will control. All publications, patent applications, patents, and other references mentioned herein are incorporated by reference. The references cited herein are not admitted to be prior art.

### Pharmaceutical Formulations

Compositions may include live or inactivated (such as by heat killing) Prevotella cells, or portions thereof, of this disclosure. The compositions may be prepared and administered as pharmaceutical formulations. The pharmaceutical formulations include Prevotella cells, or portions thereof, and at least one pharmaceutically acceptable excipient. The compositions may be formulated into a dosage form adapted for pulmonary, tracheal, or nasal administration to the subject. For example, dosage forms may include those adapted for oral or nasal inhalation, which may be to the nose, trachea, or lung(s), such as aerosols, solutions, suspensions, and dry powders.

Suitable excipients may vary depending upon the particular dosage form chosen. In addition, suitable pharmaceutically acceptable excipients may be chosen for a particular function that they may serve in the formulation. For example, certain pharmaceutically acceptable excipients may be chosen for their ability to facilitate the production of a uniform aerosol for inhalation. Alternatively or additionally, certain pharmaceutically acceptable excipients may be chosen for their ability to facilitate the production of stable dosage forms, enhance bioavailability, and/or minimize side effects.

Excipients that may be used include buffering agents, carriers, diluents, fillers, binders, disintegrants, lubricants, glidants, granulating agents, coating agents, wetting agents, solvents, co-solvents, suspending agents, emulsifiers, coloring agents, anticaking agents, humectants, chelating agents, plasticizers, viscosity agents, antioxidants, preservatives, stabilizers, and surfactants. The skilled artisan will appreciate that certain pharmaceutically acceptable excipients may serve more than one function and may serve alternative functions depending on how much of the excipient is present in the formulation and what other ingredients are present in the formulation.

In some embodiments, the Prevotella formulations may be prepared as an aerosol spray. The aerosol spray may be suitable for oral or nasal inhalation. Aerosol compositions may be in the form of a suspension or a solution and include the Prevotella compositions of this disclosure in combination with a propellant. Suitable propellants include dichlorodifluoromethane, trichlorofluoromethane, dichlorotetrafluoroethane, a hydrofluoroalkane such as tetrafluoroethane or heptafluoropropane, carbon dioxide or other suitable gas. Aerosol composition may include suitable excipients such as surfactants, e.g., oleic acid or lecithin, and/or co-solvents, e.g. ethanol. Pressurized formulations may be retained in a canister (e.g., an aluminum canister) closed with a valve (e.g., a metering valve) and fitted into an actuator provided with a mouthpiece.

In some embodiments, the Prevotella formulations may be prepared as dry powder compositions. Dry powder compositions may be suitable for topical delivery to the lung by inhalation. Dry powder compositions may be prepared as a blend of the Prevotella compositions of this disclosure and a suitable powder base such as mono-, di-or poly-saccharides (e.g., lactose or starch).

Dry powders be prepared in capsules or cartridges, such as of gelatin, or blisters, such as of laminated aluminum foil. The capsules, cartridges, or blisters may be used in a device or dispenser, such as an inhaler or insufflator. Examples of suitable devices or dispensers include a reservoir dry powder inhaler (RDPI), a multi-dose dry powder inhaler (MDPI), and a metered dose inhaler (MDI).

A reservoir dry powder inhaler (RDPI) is an inhaler having a reservoir form pack suitable for comprising multiple un-metered doses of medicament (e.g., pharmaceutical formulation) in dry powder form and including means for metering medicament dose from the reservoir to a delivery position.

A multi-dose dry powder inhaler (MDPI) is an inhaler suitable for dispensing medicament in dry powder form, wherein the medicament is located within a multi-dose pack containing (or otherwise carrying) multiple, defined doses (or parts thereof) of the Prevotella composition medicament. The multi-dose pack may be a blister pack comprising multiple blisters for containment of medicament in dry powder form. The multi-dose pack may a capsule-based pack form or a carrier onto which medicament has been applied by any suitable process including printing, painting, and vacuum occlusion.

A metered dose inhaler (MDI) is a medicament dispenser suitable for dispensing medicament in aerosol form, wherein the medicament is comprised in an aerosol container suitable for containing a propellant-based aerosol medicament formulation. The aerosol container is typically provided with a metering valve for release of the aerosol form medicament formulation to the subject. The aerosol container is generally designed to deliver a predetermined dose of medicament upon each actuation by means of the valve, which can be opened either by depressing the valve while the container is held stationary or by depressing the container while the valve is held stationary.

In some embodiments, the Prevotella formulations may be prepared as aqueous solutions or suspensions. Some solutions or suspension are suitable for inhalation by nebulization. Some solutions or suspension are suitable topical delivery to the lung by inhalation. Solutions or suspensions may be formulated with an aqueous vehicle along with one or more of a pH-adjuster (e.g., an acid, a base, a buffering salt), isotonicity-adjusting agent, and antimicrobial. In some embodiments, pharmaceutical formulations are designed for intra-nasal delivery. Such formulations may be capable of being delivered to all portions of the nasal cavities, may remain in contact with the nasal cavities for relatively long periods of time, and/or may be capable of resisting forces in the nasal passages that function to remove particles from the nose. Such formulations may be formulated with an aqueous or non-aqueous vehicle along with one or more of a thickening agent, pH-adjuster (e.g., an acid, a base, a buffering salt), isotonicity-adjusting agent, and anti-oxidant. The formulation may be applied to one nostril, such as by inhaling, while the other is manually compressed. The procedure may then be repeated for the other nostril. In some implementations, the formulation is delivered intra-nasally by use of a pre-compression pump.

The therapeutically effective concentration or dosage of cells, or portions thereof, of one or more strains of Prevotella administered to a subject may vary depending on, for example, the nature of the formulation, mode of administration, particular condition to be prevented or treated, and condition and mass of the patient. Dosage levels are typically sufficient to achieve a tissue concentration at the site of action that is at least comparable to a concentration that has been shown to be active in vitro, in vivo, ex vivo, or in tissue culture. In an example, a Prevotella composition includes live cells from one or more strains of Prevotella, and the live cells are present at about 10⁵ to about 10⁷ CFU. In an example, a Prevotella composition includes inactivated cells, such as heat-killed cells, or portions thereof, from one or more strains of Prevotella, and the inactivated cells, or portions thereof, are present at about 10⁷ CFU equivalents. The cell portions may be lipoproteins.

### Therapeutic Methods

The compositions and formulations containing cells from one or more strains of Prevotella as described herein are suitable for treating or preventing at least one symptom of a respiratory infection. A respiratory infection may be caused by caused by bacteria, viruses, or fungi. A respiratory infection may cause inflammation in one or both lungs and may cause the alveoli of the lungs to fill with fluid or pus. An example respiratory infection is bacterial pneumonia.

Administration of a Prevotella composition disclosed herein may treat bacterial pneumonia. Administration of a Prevotella composition disclosed herein may reduce one or more symptoms caused by bacterial pneumonia.

In implementations, administration of a Prevotella composition disclosed herein to a subject enhances clearance of a bacterial respiratory pathogen such as S. pneumoniae and/or S. aureus compared to a subject to which the Prevotella composition is not administered (e.g., Examples 1, 3, 12, and 13). In implementations, administration of a Prevotella composition disclosed herein improves survival of a subject exposed to a bacterial respiratory pathogen compared to a subject to which the Prevotella composition is not administered (e.g., Example 2).

Without being limited to any mechanism or mode of action, administration of a Prevotella composition disclosed herein may exert its protective effect against bacterial lung infection by inducing sub-clinical inflammation followed by inflammatory resolution. The inflammation may involve a lung-localized increase in TNFα production followed by IL-10 production. The sub-clinical inflammation may be associated with improved neutrophil killing of a bacterial pathogen. The IL-10 production may then regulate and reduce the infection-associated inflammation. See Example 11.

In implementations, at a cellular and molecular level, administration of a Prevotella composition disclosed herein may decrease serum levels of TNFα and/or may increase serum levels of cytokine IL-10 (Examples 4 and 11), each as compared to non-administration of the Prevotella composition.

In implementations, exposure to a Prevotella composition, in the absence of a bacterial infection, induces a pro-inflammatory response in the lung (e.g., Example 5). The pro-inflammatory response may include increased neutrophil recruitment and activation. Accordingly, the Prevotella compositions disclosed herein may be used to activate, enhance, and/or promote an innate immune response in a subject, with or without a respiratory infection.

In implementations, exposure to a Prevotella composition increases the number of neutrophils recruited to the lungs in response a respiratory infection (e.g., Example 6). In implementations, exposure to a Prevotella composition causes TNFα production by neutrophils (e.g., Example 6).

In implementations, exposure to a Prevotella composition in a subject without an intact microbiome provides protection against a bacterial respiratory pathogen (e.g., Example 7).

In implementations, exposure to a Prevotella composition activates TLR2-dependent neutrophil recruitment (e.g., Example 9) and secretion of TNFα in neutrophils (e.g., Examples 8 and 12). The neutrophils may kill more bacterial pathogen cells than in the absence of the Prevotella-mediated activation (e.g., Examples 10 and 13). The neutrophil killing may be serine protease-mediated (e.g., Example 10).

The formulations of this disclosure can be administered to a subject before or after onset of bacterial pneumonia. The frequency and duration of administration of a Prevotella composition may vary. In embodiments, an effective amount of a Prevotella composition may be administered once a day for one or two days. In embodiments, an effective amount of a Prevotella composition may be administered twice daily for a two-week treatment period. Doses may be administered more than once or twice a day, such as three times per day. Doses may be administered on a weekly basis, for example one, two, three, four, five, six, or more times per week. Monthly administrations may also be implemented, such that a Prevotella composition is administered one, two, three, four, or more times per month.

The number of times per day, week, or month that the disclosed formulations are administered to a subject, along with the entire duration of the treatment period, may depend on the severity or type of condition a subject is experiencing or is expected to experience. For example, embodiments in which a Prevotella composition is administered to treat existing bacterial pneumonia may involve more frequent administrations than embodiments in which a Prevotella composition is administered to prevent bacterial pneumonia. Embodiments in which a Prevotella composition is administered to prevent bacterial pneumonia may involve a longer treatment period than embodiments in which a Prevotella composition is administered to treat existing bacterial pneumonia. For example, as a prophylactic immunobiotic, a Prevotella composition may be taken daily for an indefinite period similar to a probiotic for gut health. As a therapeutic, a Prevotella composition may be taken daily until the bacterial pneumonia infection clears, such as for one week. The length of the treatment period may also be patient-specific and re-evaluated periodically by a physician or other health care provider.

### EXAMPLES

The following examples illustrate various aspects of the disclosure, and should not be considered limiting.

### Methods

In the examples below, adult male and female mice aged 6-12 weeks were used as follows. C57BL/6 J (wild-type, “WT”), B6.129Tlr2tm1Kir (“Tlr2−/−”), and B6.129il10tm1Cgn (“Il10−/−”) mice were purchased from Jackson Laboratory (stocks #000664, 004650, and 002251, respectively). All strains used in the examples (WT, Tlr2−/− and Il10−/−) are on the C57BL/6J genetic background. Mice were maintained in the University of Colorado Office of Laboratory Animal Resources. Housing conditions included a light cycle of 14:10 (light: dark) hours, a temperature of 72±2° F., and 4 ±10% humidity. Mice were fed irradiated Tecklad diet (Envigo, Inotiv, Inc.; catalog #2920X for colony mice; catalog #2919 for breeder pairs). Germ-free mice were obtained from the University of Colorado Anschutz Medical Campus Gnotobiotic Facility, which maintains a colony established with founder C57BL/6 mice obtained from the National Gnotobiotic Rodent Resource Center at the University of North Carolina. Germ-free mice were housed in sterilized vinyl film isolators with positive pressure air flow through HEPA filtration. Any items introduced into the isolators were sterilized, with quality control indicators to verify sterilization. The internal isolator environment and housed mice were tested bi-weekly and prior to experimental use for microbiota through culture-dependent methods and by qPCR (see depletion of microbiomes in the following paragraph). For infection experiments, germ-free mice were transferred directly from the Gnotobiotic Core Facility into BSL2 vivarium space. Transferred mice were exposed to input bacteria as described in the applicable examples, below, within 8 hours of transfer.

In some of the following examples, the microbiomes of mice were depleted. Antibiotic-treated mice were exposed to a broad-spectrum antibiotic cocktail (ampicillin 1 g/L, neomycin 1 g/L, metronidazole 1 g/L, vancomycin 0.5 g/L, MilliporeSigma and Mckesson) in drinking water ad libitum for 7 days. Water containing antibiotics was replaced with normal drinking water 48 hours prior to live Prevotella exposure. Microbiome depletion was confirmed by qPCR using genomic DNA extracted from stool samples using the PureLink™ Genomic DNA Mini Kit (ThermoFisher Scientific). Primers (ACTCCTACGGGAGGCAGCAGT and ATTACCGCGGCTGCTGGC) were used with iTaq™ Universal SYBR® Green Supermix (BioRad) and 1 μL template DNA, with reactions performed on a CFX Connect™ Real-Time System (BioRad) under the following cycle conditions: (1) 94° C. for 4 minutes; (2) 40 cycles of 15 seconds at 95° C., 30 seconds at 60° C., and (3) 72° C. for 10 minutes. Total 16S rRNA gene copy numbers were calculated using a standard curve generated with a known concentration of S. pneumoniae D39 gDNA, input ng/μL DNA, and Ct values. qPCR data were analyzed using CFX Manager Software (version 2.1, BioRad).

The following Examples include use of flow cytometry, performed as follows. Lungs were harvested following perfusion by transcardial injection of 10 mL PBS, and single cells were prepared for flow cytometry. Briefly, lungs were subjected to mechanical (mincing) and enzymatic (DNAseI 30 μg/mL, Sigma, and type 4 collagenase 1 mg/mL, Worthington Biochemical Corporation) digestion prior to passage through a 70 μM strainer. Red blood cells were lysed in RBC lysis buffer (0.15M NH4Cl, 10 mM KHCO₃, 0.1 mM Na₂EDTA, pH 7.4). Fc receptors were blocked by incubation in anti-CD16/32 (2.4G2 hybridoma supernatant) prior to staining in FACS buffer (1% BSA, 0.01% NaN₃, PBS). For intracellular flow cytometry, cells were incubated with Brefeldin A (BD Biosciences) prior to staining and permeabilized with 1 mg/mL saponin (Sigma) prior to intracellular staining. All cells were fixed in 1% paraformaldehyde. Antibodies used for staining included the following anti-mouse antibodies: Siglec F (BD, catalog #562681, clone E50-2440, lot #B302914), MHCII (BioLegend, catalog #107643, clone M5/114.15.2, lot #B317262), Ly6G (BioLegend, catalog #127614, clone 1A8, lot #B292772), Ly6C (BioLegend, catalog #128012, clone HK1.4, lot #B250462), CD45.2 (BD, catalog #564616, clone 104, lot #1083734), CD11 c (BioLegend, catalog #117338, clone N418, lot #B290360), CD11b (BioLegend, catalog #101212, clone M1/70, lot #B281906), and TNFα (ThermoFisher Scientific, catalog #25-7321-82, clone MP6-XT22, lot #2044683). All antibodies were used at a 1:200 dilution for staining. Flow cytometry was performed on an LSR Fortessa X-20 in the ImmunoMicro Flow Cytometry Shared Resource Laboratory at the University of Colorado Anschutz Medical Campus (RRID: SCR_021321). Data analysis was performed using FlowJo™ Software, version 9.9.6 (BD Life Sciences).

Bronchoalveolar lavage fluid (BAL) cytokines and chemokines with the exception of MIP-2 were measured using a LEGENDplex™M Mouse Inflammation Panel (BioLegend), with analytes detected on the LSR Fortessa X-20 in the ImmunoMicro Flow Cytometry Shared

Resource Laboratory at the University of Colorado Anschutz Medical Campus (RRID: SCR_021321). Data were analyzed using the LEGENDplex™ Data Analysis Software Suite (BioLegend). BAL MIP-2 was measured using a mouse CXCL2/MIP-2 ELISA kit (R&D Systems), serum cytokines were measured using mouse IL-10 and TNFα ELISA kits (BD), and analytes were detected on a Synergy™ HT Microplate Reader (BioTek). Data were analyzed using Prism (GraphPad, version 8).

For cell isolations and stimulations in the following examples, bone marrow neutrophils were isolated from the femurs of mice by Histopaque density gradient centrifugation and purity was confirmed to be >80% Ly6G+neutrophils by flow cytometry. P. melaninogenica lipoproteins were prepared by Triton X-114 phase partitioning and P. melaninogenica lipopolysaccharide (“LPS”) was prepared using an LPS isolation kit (Sigma). Concentrations were determined relative to Pam3SK4 and E. coli LPS standard curves by running purified lipoprotein and LPS preparations on a 15% sodium dodecyl-sulfate polyacrylamide gel electrophoresis gel prior to detection using a Silver Stain Kit (Bio-Rad Laboratories, Inc) and imaging on a ChemiDoc XRS+Gel Imaging System (Bio-Rad Laboratories, Inc). P. melaninogenica LPS endotoxin activity was confirmed using a Pierce™ Chromogenic Endotoxin Quant Kit (ThermoFisher Scientific). Lipoprotein lipase-treated P. melaninogenica heat killed (“HK”) was prepared by incubation with 200 μg lipoprotein lipase (Sigma) for 2 hours at 37° C. For cell stimulation assays, 10⁵ neutrophils in RP10 media supplemented with penicillin and streptomycin were exposed to P. melaninogenica HK (1:1 ratio), lipase-treated P. melaninogenica HK, P. melaninogenica lipoprotein (10 ng/mL), P. melaninogenica LPS (10 ng/mL), C29 TLR2 inhibitor (100-200 μM, Selleck Chemicals), Resatorvid TAK-242 TLR4 inhibitor (100-200 μM, Selleck Chemicals), or Pam3SK4 (10 ng/mL, InvivoGen, San Diego, CA) and incubated for 24 hours at 37° C. with 5% CO2 prior to supernatant collection. For cell infection assays, 10⁵ neutrophils were exposed to 10⁵ S. pneumoniae for 1 hour prior to washing and incubation for 24 hours at 37° C. with 5% CO₂ with or without P. melaninogenica HK (1:1 ratio) in media containing gentamycin (10 μg/mL).

For neutrophil functional assays in the following examples, lung neutrophils were isolated by positive selection (MojoSort PE-positive selection kit, BioLegend), and purity was confirmed to be >90% Ly6G+neutrophils by flow cytometry. Similar results were obtained using lung neutrophils isolated by negative isolation (19762, Mouse Neutrophil Enrichment kit, STEMCELL Technologies). Neutrophils were isolated from the lungs of mice following 24-hour treatment with PBS, E. coli LPS, or P. melaninogenica HK intratracheally (“i.t.”) as indicated. For opsonophagocytic killing assays, 10³ S. pneumoniae grown to mid-log phase were opsonized for 30 minutes with 3% fresh mouse sera prior to incubation with 10⁵ neutrophils in Hank's buffer/0.1% gelatin for 1 hour at 37° C. under rotation. Killing assays were completed in the presence or absence of 1× Halt™ protease inhibitor cocktail (ThermoFisher Scientific) or 10 μM diphenyleneiodonium chloride (DPI), (ThermoFisher Scientific), which did not impact neutrophil viability over the course of 1 hour. Colony forming units (“CFU”) were measured by serial dilution plating, and percent killing was determined relative to reactions without neutrophils. Serine protease activity was determined using substrates specific to elastase (0.85 mM MeOSuc-Ala-Ala-Pro-Val-pNA, Sigma) and cathepsin G (0.1 mM Succinyl-Ala-Ala-Pro-Phe-pNA, Sigma). Briefly, 10⁵ purified neutrophils were incubated with or without 1× Halt™ protease inhibitor cocktail for 30 minutes prior to washing and lysis in 0.1% Triton X-100. Substrates were added to neutrophil lysates and incubated in the dark for 45 minutes at 37° C. Absorbance was determined by reading the OD410 on Synergy™ HT Microplate Reader (BioTek) minus control wells with no neutrophils added. For detection of total reactive oxygen species (“ROS”), 10⁵ purified neutrophils were resuspended in KRP buffer (5 mM glucose, 1 mM CaCl₂, 1 mM MgSO₄ in PBS) and equilibrated for 15 minutes prior to plating on Greiner Bio-One LUMITRAC™ plates (FisherScientific) followed by the addition of 50 μM luminol (FisherScientific). Luminescence over 1 hour at 37° C. was detected using a Synergy™ HT Microplate Reader (BioTek). Area under curve (AUC) was calculated, minus wells with no neutrophils added.

All measurements for the following examples were taken from distinct samples, with exact sample sizes indicated below. Prism (GraphPad, version 8) was used for all statistical analyses. All data were tested for normality using the Shapiro-Wilk test. For data with normal distributions, two-tailed Student's t-tests, one-way or two-way ANOVA tests with Dunnett's, Sidak's, or Tukey's post hoc analyses for multiple comparisons were used as specified. Two-tailed Mann-Whitney U-tests and Kruskal-Wallis tests with Dunn's post hoc analysis for multiple comparisons were used for all data with non-Gaussian distributions. Logrank Mantel-Cox test was used for survival group comparison. For all statistical tests, p-values <0.05 were considered significant. Individual p-values are specified in the following examples. In the figures, “LOD”=limit of detection.

### Example 1: Effects of Live P. melaninogenica on Clearance of S. pneumoniae From the Murine Lung

Adult male and female C57BL/6J mice were maintained as described above. Prevotella melaninogenica strain ATCCR 25845™ (American Type Culture Collection) was grown under anaerobic conditions on Brucella Agar containing 5% sheep blood, hemin, and vitamin K at 37° C. for 72 hours. A streptomycin-resistant variant of serotype 2 Streptococcus pneumoniae strain D39 (gifted from New York University) was grown in Todd Hewitt Broth with 5% Yeast Extract (BD Bacto™), with 50 μg/mL streptomycin (Sigma) at 37° C. with 5% CO₂ without shaking. When Streptococcus was grown for the following Examples, streptomycin was included only for the streptomycin-resistant D39 strain. Although the murine airway microbiome contains both Prevotella and Streptococcus species, the foregoing strains are not resident members, which allowed for controlled exposure to each bacterium.

Bacterial suspensions from fresh plates of P. melaninogenica were prepared in PBS to an optical density (OD600) of 0.3, centrifuged at ≥20,000× g for 10 min, and re-suspended in PBS prior to injection. S. pneumoniae was grown in broth from frozen stocks to mid-log phase and centrifuged at ≥20,000× g for 10 min followed by resuspension in PBS for infections. Inoculum burdens were determined by serial dilution for colony forming units (CFU) enumeration.

To model Prevotella aspiration, live P. melaninogenica (or PBS control) was instilled intratracheally (“i.t.”) in a volume of 50 μL on mice anesthetized by inhaled isoflurane. S. pneumoniae challenge at 5×10⁶ CFU/mouse (or PBS control) began 24 hours later and lasted for 24 hours. Lungs were collected and homogenized using a Bullet Blender tissue homogenizer (Stellar Scientific, Baltimore, MD). S. pneumoniae burdens were calculated following serial dilution in PBS and growth on Tryptic Soy agar plates containing neomycin (5 μg/mL, Sigma) and streptomycin (50 μg/mL) prepared with fresh catalase (5000 units/plate, Worthington Biochemical Corporation, Lakewood, NJ). Plates were grown at 37° C. with 5% CO₂ for 24 hours.

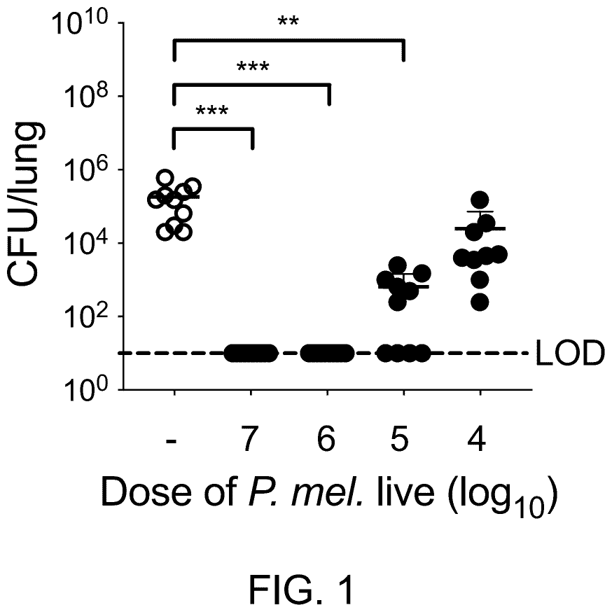

Results are shown in FIG. 1. Data are pooled from three independent experiments, displayed as mean±SEM, n=10 mice/group. From left to right, ***p<0.0001, ***p<0.0001, **p=0.0015, Kruskal-Wallis with Dunn's post hoc test.

S. pneumoniae burdens at 24 hours post-infection were not detected in any mice pre-exposed to an equivalent dose of live P. melaninogenica, compared to burdens of ˜10⁵ CFU in mice infected with S. pneumoniae alone. Titration of P. melaninogenica revealed that significant protection was maintained with up to a 100-fold lower dose of live P. melaninogenica than that of S. pneumoniae. The results demonstrate that exposure to live P. melaninogenica mediates rapid clearance of S. pneumoniae from the murine lung.

### Example 2: Effects of Heat-Killed P. melaninogenica on Murine Survival Following S. pneumoniae Challenge

Experiments were performed as described for Example 1 except that inactivated instead of live Prevotella was used, and the administered S. pneumoniae dose was lethal (10⁷ CFU/mouse). An equivalent dose (i.e., 10⁷ CFU equivalents/mouse) of inactivated Prevotella was used. In the following examples that utilized heat-killed (“HK”) Prevotella, 10⁷ CFU equivalents/mouse were used.

HK Prevotella were prepared from fresh plates following resuspension in PBS and incubation at 56° C. for 35 minutes. Samples before and after heat-killing were used to determine CFU equivalents/mL and confirm killing, respectively.

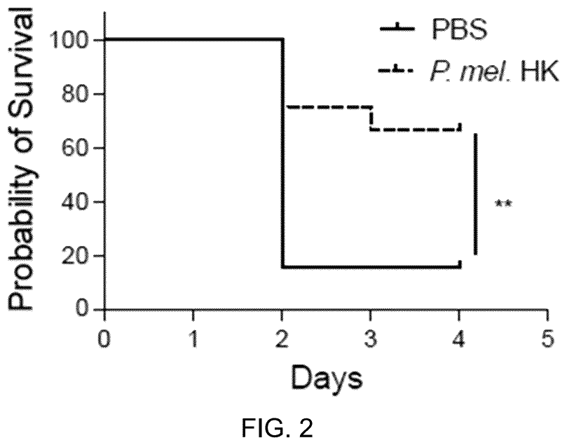

Results are shown in FIG. 2, in which **p=0.0075, Mantel-Cox survival test, and n=13 mice/group.

Exposure to P. melaninogenica significantly increased the probability of survival, with 33% of Prevotella exposed mice succumbing to infection compared to 85% of mice infected with S. pneumoniae alone. Improved survival in mice exposed to P. melaninogenica correlated with lower pneumococcal burdens in the lung three days post-infection (data not shown). The rapid protection induced by P. melaninogenica (see FIG. 1) was not dependent on instillation of Prevotella into the lung, as intranasal inoculation similarly enhanced S. pneumoniae clearance by 24 hours (data not shown). Also, P. melaninogenica was protective against both serotype 2 S. pneumoniae, which spreads systemically by 24 hours, and serotype 3 S. pneumoniae, which is restricted to the lung (data not shown). Together, the results presented in Examples 1 and 2 indicate that exposure to live or inactivated P. melaninogenica protects against S. pneumoniae infection.

### Example 3: Effects of Heat-Killed Bacteria on Clearance of S. pneumoniae From the Murine Lung

Additional heat-killed bacterial species were investigated to determine if installation of any HK bacterium enhances protection against S. pneumoniae. Experiments were performed as in Examples 1 and 2 with the following modifications.

Heat-killed Corynebacterium accolens strain ATCCR 49726™ (American Type Culture Collection) and Corynebacterium amycolatum strain SK46 (catalog #HM-109, BEI resources, NIAID, NIH as part of the Human Microbiome project) were prepared following growth in BHI broth cultures supplemented with 1% Tween® 80 (polysorbate, VWR). Heat-killed Streptococcus salivarius strain SK126 (catalog #HM-109, obtained from BEI Resources, NIAID, NIH as part of the Human Microbiome project) was prepared following growth in Todd Hewitt Broth with 5% Yeast Extract (BD Bacto™) at 37° C. with 5% CO₂ without shaking. All three species are Gram-positive and are common members of the upper airway microbiota.

None of HK C. accolens, C. amycolatum, or S. salivarius were protective (data not shown). The results indicate that airway commensals generally, compared to P. melaninogenica specifically, do not have a protective effect against S. pneumoniae lung infection.

P. melaninogenica is a Gram-negative bacterium, so the effects of another Gram-negative bacterium, Escherichia coli, was investigated. Heat-killed E. coli (strain DH5α, ThermoFisher Scientific) was prepared following growth in LB broth (BD Bacto™) at 37° C. with shaking (200 rpm). E. coli lipopolysaccharide (LPS) (0111: B4, Sigma) was also investigated by treating mice with 10 μg (LPS) intratracheally 24 hours prior to S. pneumoniae infection.

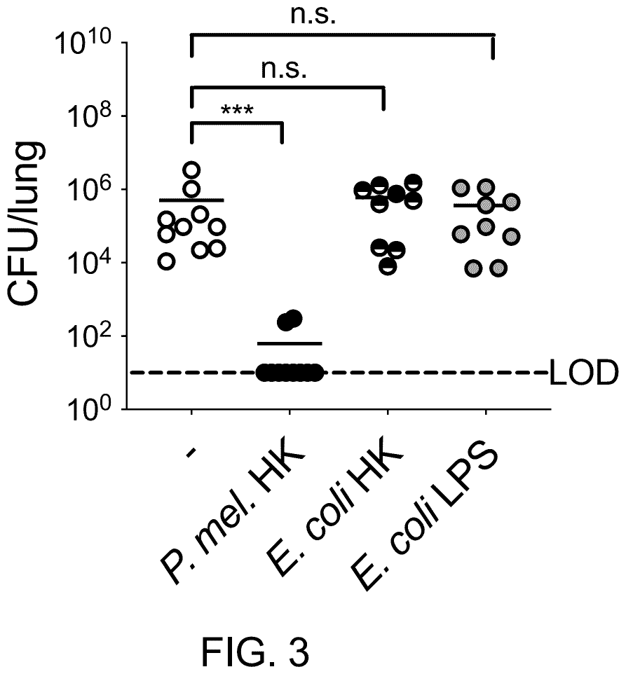

Results are shown in FIG. 3 and depict lung S. pneumoniae burdens in mice following exposure to PBS (-), P. melaninogenica (“P. mel.”) HK, E. coli HK, or E. coli lipopolysaccharide (LPS) i.t. prior to a 24-hour S. pneumoniae infection at 5×10⁶ CFU/mouse (n=10 mice/group). From left to right, ***p=0.0008, p>0.9999, p>0.9999, Kruskal-Wallis with Dunn's post hoc test.

The results demonstrate that, unlike P. melaninogenica, neither E. coli HK nor E. coli LPS enhanced clearance of S. pneumoniae from the lung. The results indicate that Gram-negative bacteria generally, compared to P. melaninogenica specifically, do not have a protective effect against S. pneumoniae lung infection. LPS is a known immune stimulator and is used in some following Examples as an immune stimulatory but non-protective comparative tool.

### Example 4: Effects of Heat-Killed Bacteria on Serum Cytokines

The mice of Example 3 were also evaluated for serum cytokines using mouse IL-10 and TNFα ELISA kits (BD).

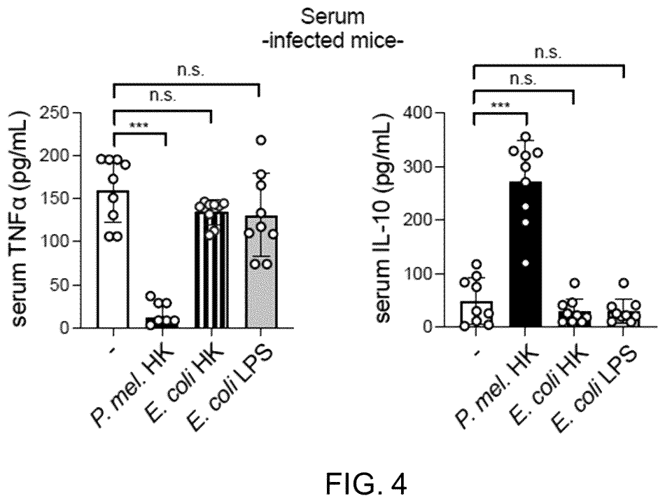

Results are shown in FIG. 4 and depict IL-10 and TNFα in mice following exposure to PBS (-), P. melaninogenica (“P. mel.”) HK, E. coli HK, or E. coli lipopolysaccharide (LPS) i.t. prior to a 24-hour S. pneumoniae infection at 5×10⁶ CFU/mouse (n=10 mice/group). From left to right for TNFα: ***p<0.0001, p=0.2352, p=0.1558, and for IL-10: ***p<0.0001, p=0.6977, p=0.7136, one-way ANOVA with Dunnett's post hoc test.

The results demonstrate that P. melaninogenica HK significantly reduced serum levels of the pro-inflammatory cytokine TNFα compared to control, but E. coli HK or LPS did not. P. melaninogenica HK significantly increased serum levels of cytokine IL-10, but E. coli HK or LPS did not.

### Example 5: Effects of Heat-Killed P. melaninogenica on the Innate Immune System

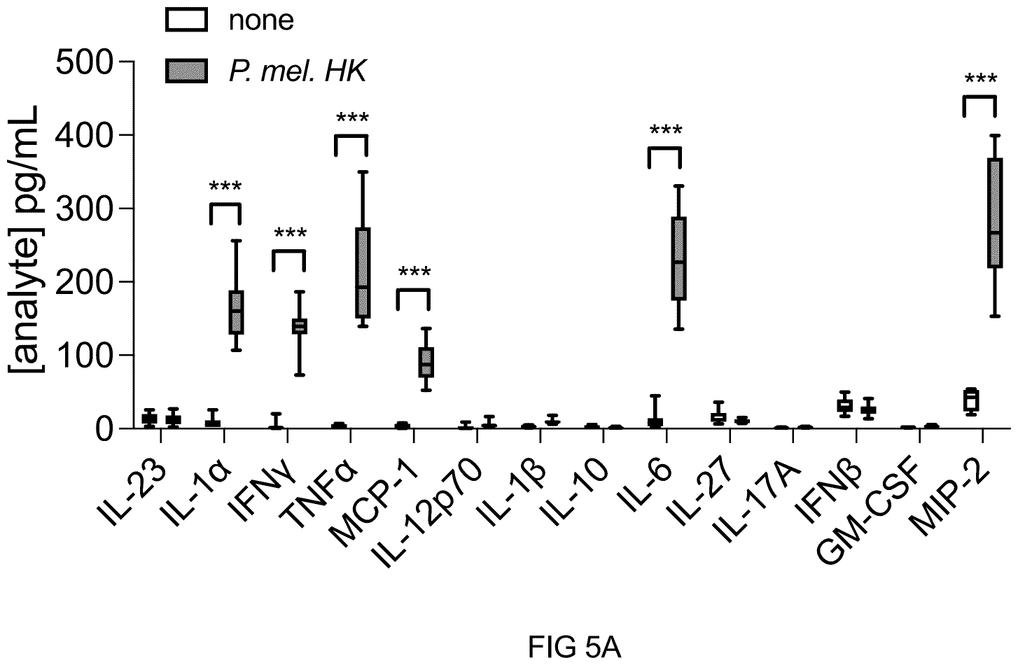

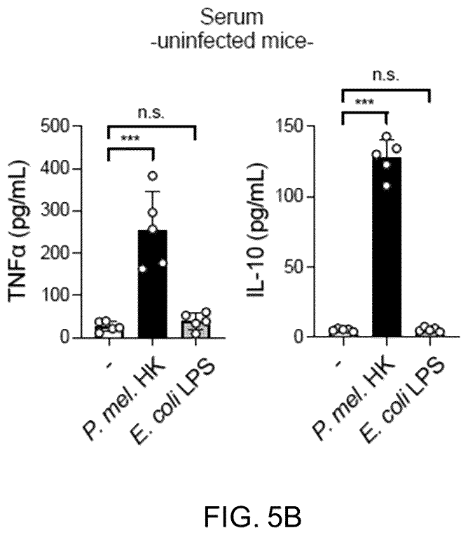

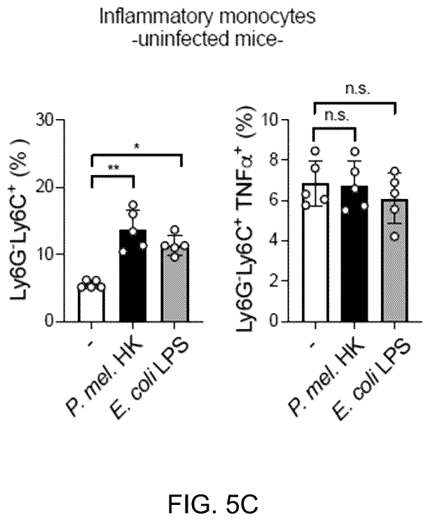

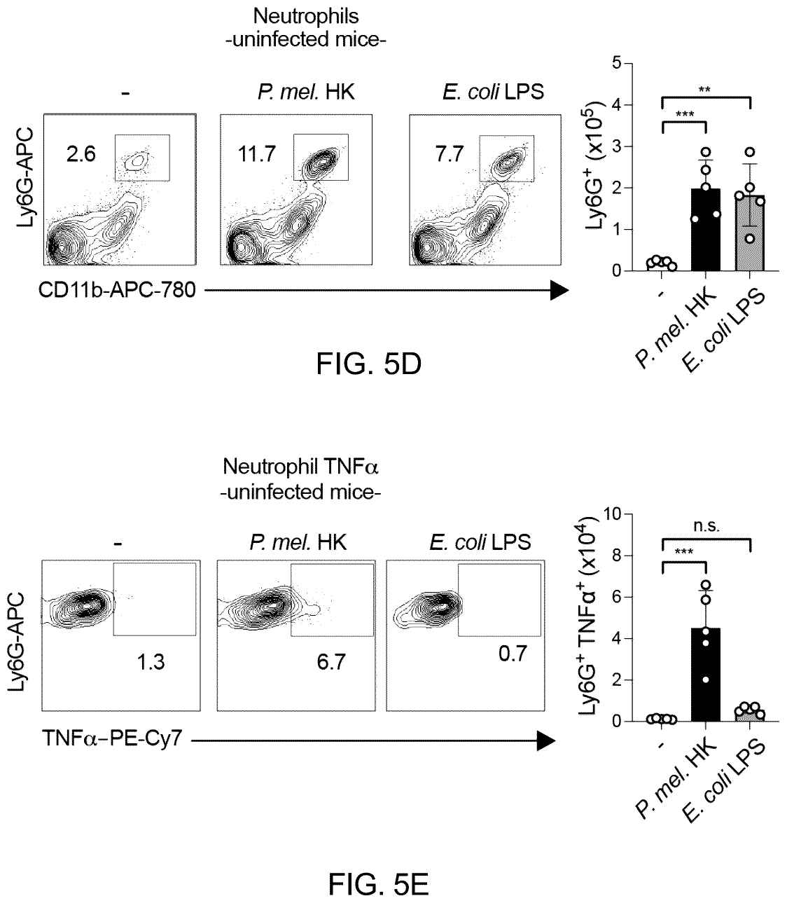

The effect of P. melaninogenica on the innate immune system was investigated. First, the immune response induced by P. melaninogenica HK (strain 25845), E. coli LPS, or PBS (as a negative control, “-”) was examined in the absence of S. pneumoniae infection (i.e., “uninfected mice”). Ten (FIG. 5A) or five (FIGS. 5B-5E) mice per group were studied. The levels of various cytokines and chemokines in lung bronchoalveolar lavage fluid (BAL) or serum following intratracheal exposure to a stimulus or control for 24 hours as described in Example 1 were investigated.

Results are shown in FIGS. 5A-5E. P. melaninogenica exposure increased the production of several pro-inflammatory cytokines in BAL, including TNFα, IL-6, IL-1α, and IFNγ as well as the chemokines MCP-1 (CCL2) and MIP-2 (CXCL2), a major neutrophil chemoattractant (FIG. 5A).

P. melaninogenica also increased systemic TNFα and IL-10 compared to mice treated with PBS or E. coli LPS (FIG. 5B). The lungs of the same mice were prepared for intracellular flow cytometry as described above. P. melaninogenica significantly enhanced the recruitment of myeloid cells including inflammatory monocytes (CD45⁺SiglecF⁻Ly6G⁺Ly6C⁺CD11b⁺ cells) and neutrophils (CD45⁺SiglecF⁻Ly6G⁺CD11b⁺ cells), similar to E. coli LPS (FIGS. 5C & 5D). P. melaninogenica also induced TNFα production in lung neutrophils, but E. coli LPS did not (FIG. 5E). Neither P. melaninogenica nor E. coli LPS affected the recruitment or TNFα production of CD11bhi dendritic cells (DCs) (data not shown).

Together, these data indicate that exposure to P. melaninogenica, in the absence of S. pneumoniae infection induces a pro-inflammatory response in the lung associated with increased neutrophil recruitment and activation. Also, innate immune cell activation profiles for mice exposed to the protective P. melaninogenica are different than for mice exposed to the stimulatory E. coli LPS.

Data are pooled from three independent experiments (FIG. 5A) or representative from one of four independent experiments (FIGS. 5B-5E). Box boundaries in FIG. 5A indicate the 25th and 75th percentiles, with a horizontal line representing the median and whiskers indicating minimum and maximum values. Data in FIGS. 5B-5E are displayed as mean±SEM. For FIG. 5A, ***p<0.0001, two-way ANOVA with Sidak's post hoc test. For FIG. 5B from left to right, ***p<0.0001, p=0.9108 (TNFα), ***p<0.0001, p=0.9108 (IL-10), oneway ANOVA with Dunnett's post hoc test and from left to right p=0.0060, p>0.9999 (TNFα), p=0.0144, p>0.9999 (IL-10), Kruskal-Wallis with Dunn's post hoc test. In FIG. 5C from left to right, **p=0.0044, *p=0.0453, p=0.9875, p=0.5395, Kruskal-Wallis with Dunn's post hoc test. In FIG. 5D from left to right ***p=0.009, **p=0.0017, one-way ANOVA with Dunnett's post hoc test and p=0.0115, p=0.0215, Kruskal-Wallis with Dunn's post hoc test. In FIG. 5E from left to right, ***p<0.0001, p=0.7296, one-way ANOVA with Dunnett's post hoc test and p=0.0008, p=0.1542, Kruskal Wallis with Dunn's post hoc test.

### Example 6: Roles of Neutrophils and TNFα in P. melaninogenica-Mediated Protection

Neutrophils are known to be involved in S. pneumoniae killing at early time points, prior to the development of specific immunity, which is typically required for infection clearance. Accordingly, the role of neutrophils and TNFα in P. melaninogenica-mediated protection from S. pneumoniae infection was investigated. Experiments included depleting neutrophils prior to S. pneumoniae infection, as described above. Experiments included depleting TNFα by treating mice intraperitoneally (i.p.) 24 hours prior to S. pneumoniae infection with 200 μg/mouse isotype control anti-IgG2A antibody (clone C1.18, catalog #BE0085, lot #722719J2), anti-Ly6G antibody (clone 1A8, catalog #BE0071-1, lot #80772101), or anti-TNFα antibody (clone XT3.11, catalog #BE0058, lot #728221A1).

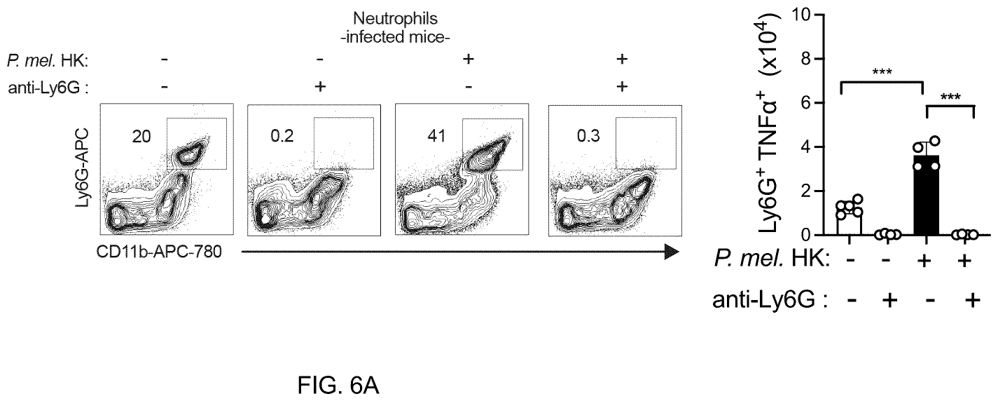

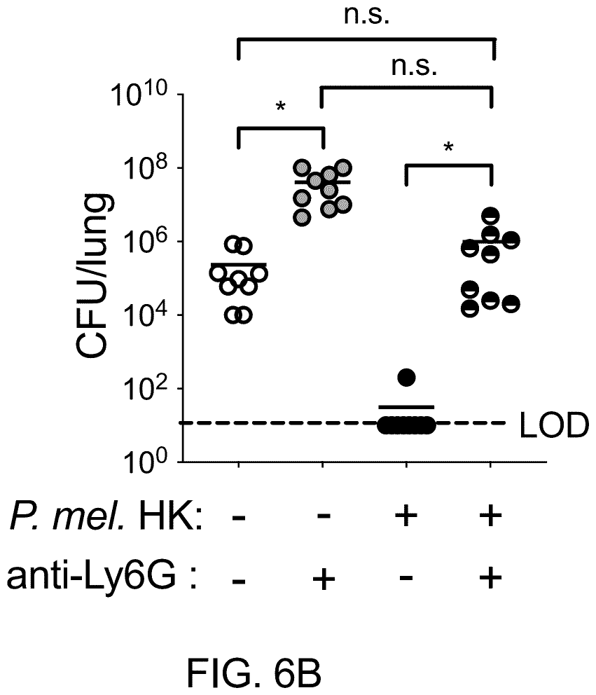

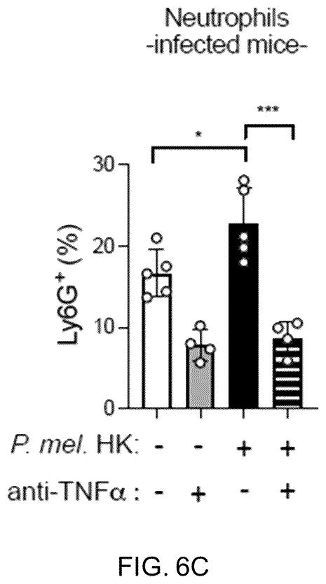

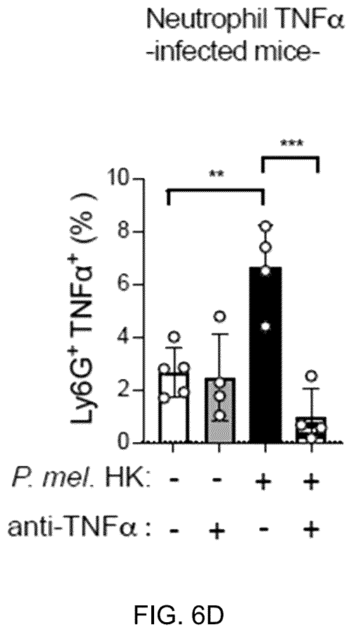

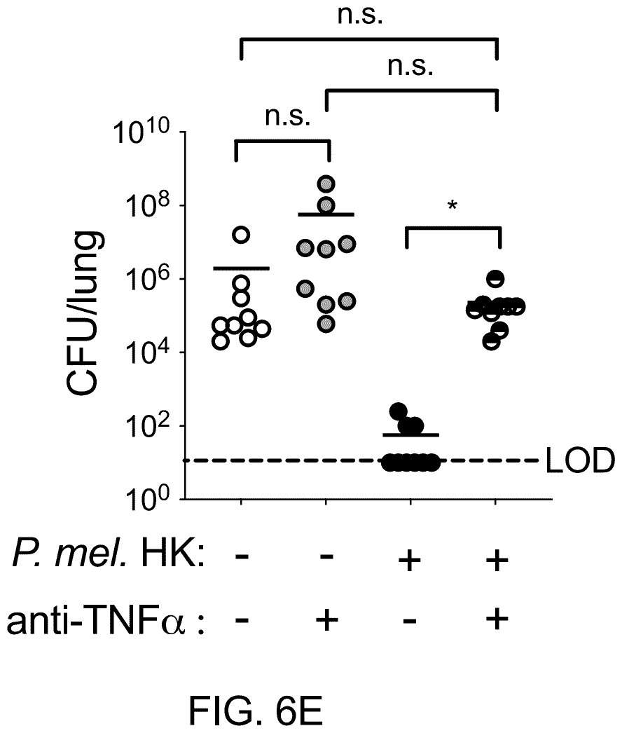

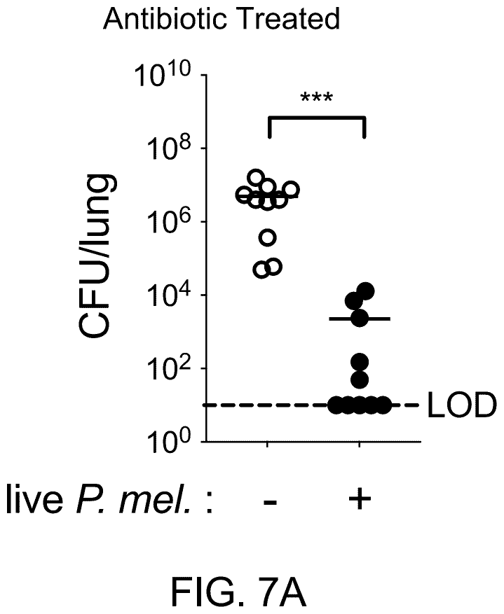

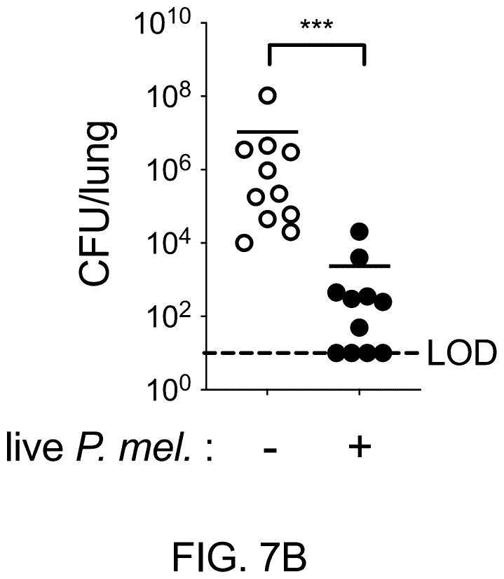

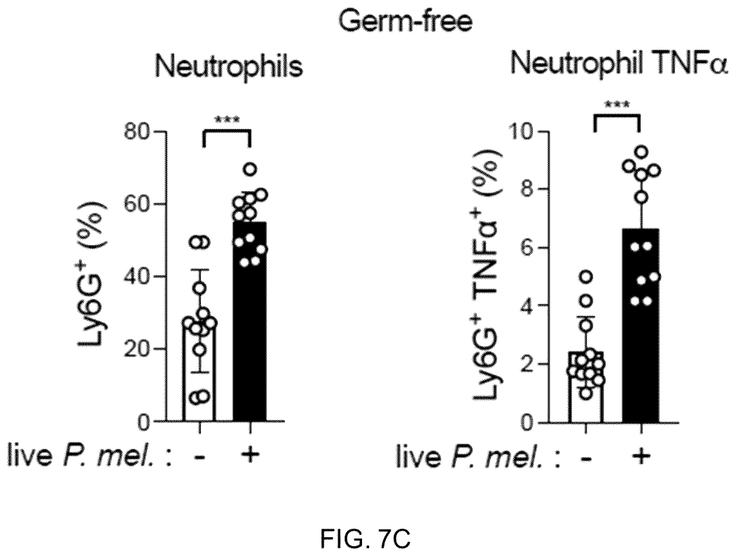

Results are shown in FIGS. 6A-6E. Exposure to P. melaninogenica HK increased the number of neutrophils recruited to the lungs in response to S. pneumoniae infection (FIG. 6A). Following neutrophil depletion, P. melaninogenica was no longer protective against S. pneumoniae, as lung burdens in Prevotella-exposed mice were similar to those in mice infected with S. pneumoniae alone (FIG. 6B). The results demonstrate that P. melaninogenica's protection against S. pneumoniae is mediated by neutrophils.

The lungs of neutrophil-depleted mice also showed a significant reduction in TNFα (data not shown), suggesting neutrophils as a major source of TNFα following P. melaninogenica exposure and S. pneumoniae infection. In TNFα-depleted mice, neutrophil recruitment and production of TNFα were reduced (FIGS. 6C & 6D). Similar to the effect of neutrophil depletion (FIG. 6B), TNFα depletion resulted in loss of P. melaninogenica-mediated protection (FIG. 6E). Inflammatory monocyte recruitment was also reduced in TNFα-depleted mice, though CD11bhi DCs and AM populations were unchanged (data not shown). Together, these data indicate a role for both neutrophils and TNFα in P. melaninogenica-mediated protection against S. pneumoniae.

FIGS. 6A & 6B show representative flow cytometry plots and total cell numbers for neutrophils detected by flow cytometry (n=5 mice/group, PBS; n=4 mice/group, others) (FIG. 6A) and lung burdens of type 2 S. pneumoniae (n=9 mice/group) (FIG. 6B) in mice treated with isotype control or anti-Ly6G antibodies (200 μg/mouse) i.p. together with either PBS (-) or P. mel. strain 25845 HK i.t. prior to 24 hour S. pneumoniae infection at 5×10⁶ CFU/mouse. FIGS. 6C-6E show total cell number of neutrophils and neutrophil TNFα detected by intracellular flow cytometry (n=5 mice/group for isotype control groups, 4 mice/group for anti-TNFα groups) (FIGS. 6C & 6D) and lung S. pneumoniae burdens (n=9 mice/group) (FIG. 6E) in mice treated with isotype control or anti-TNFα antibodies (200 μg/mouse) i.p. together with either PBS (-) or P. mel. HK i.t. prior to 24 hour S. pneumoniae infection. Data are pooled from three independent experiments (FIGS. 6B & 6E) or representative from one of four independent experiments (FIGS. 6A, 6C, & 6D), displayed as mean±SEM. For FIG. 6A, ***p<0.0001, one-way ANOVA with Sidak's post hoc test. For FIG. 6B from left to right, *p=0.0222, p>0.9999, p=0.0751, *p=0.0192, Kruskal-Wallis with Dunn's post hoc test. In FIG. 6C, from left to right, *p=0.0337, ***p<0.0001 one-way ANOVA with Tukey's post hoc test; in FIG. 6D **p=0.003, ***p=0.0002 one-way ANOVA with Tukey's post hoc test; and in FIG. 6E, from left to right, p=0.3058, p>0.9999, p=0.4470, *p=0.0118, Kruskal-Wallis with Dunn's post hoc test.

### Example 7: Effects of P. melaninogenica on Microbiome-Depleted Mice

Results from the foregoing Examples suggest that innate immune priming is involved in P. melaninogenica-mediated protection from S. pneumoniae infection, but not whether the endogenous microbiome, which helps regulate immune homeostasis, is required for this protective effect. The contribution of the endogenous microbiome to protection in Prevotella-exposed mice was evaluated using antibiotic-treated (i.e., microbiome-depleted) and Germ-free mice, which are described above.

Results are shown in FIGS. 7A-7C. FIG. 7A shows lung S. pneumoniae burdens in mice treated with antibiotics followed by exposure to PBS (-) or live P. mel. strain 25845 i.t. prior to 24 hour S. pneumoniae infection at 5×10⁶ CFU/mouse (n=9 mice/group). FIGS. 7B & 7C show lung S. pneumoniae burdens (FIG. 7B) and percent neutrophils and neutrophil TNFα (FIG. 7C) in Germ-free mice treated with either PBS (-) or live P. mel. i.t. prior to 24 hour S. pneumoniae infection at 10⁶ CFU/mouse (n=11 mice/group). Data are pooled from three independent experiments and are displayed as mean±SEM.

In antibiotic-treated mice, live P. melaninogenica significantly (***p<0.0001, two-tailed Mann-Whitney U-test) improved S. pneumoniae clearance from the lungs by 24 hours (FIG. 7A). Similarly, exposure to live P. melaninogenica significantly (***p<0.0001, two-tailed Mann-Whitney U-test) enhanced clearance of S. pneumoniae from the lungs of Germ-free mice (FIG. 7B). In Germ-free mice, Prevotella-mediated protection against S. pneumoniae was associated with significantly (***p<0.0001, two-tailed t-test) increased lung neutrophil recruitment (FIG. 7C, left side) and activation (FIG. 7C, right side). Together, these data demonstrate that P. melaninogenica is sufficient to improve protection against S. pneumoniae lung infection in the absence of an intact microbiome.

### Example 8: Role of TLR2 in P. melaninogenica-Induced TNFα (In Vitro)

In this example, the host and bacterial requirements for P. melaninogenica-induced neutrophil TNFα in vitro were investigated as described in more detail below.

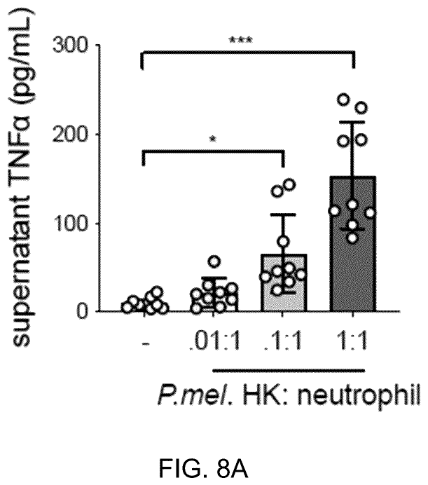

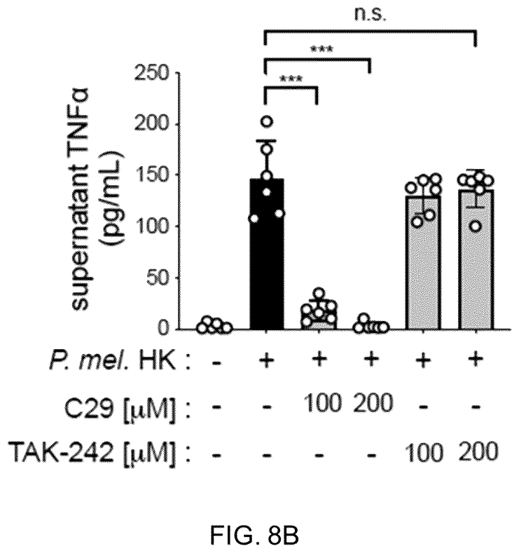

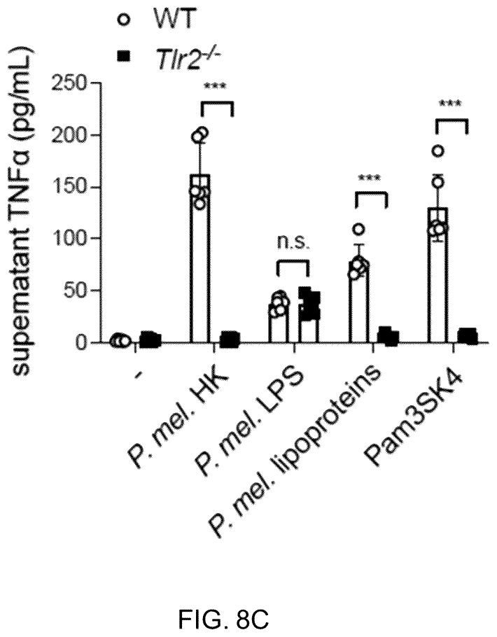

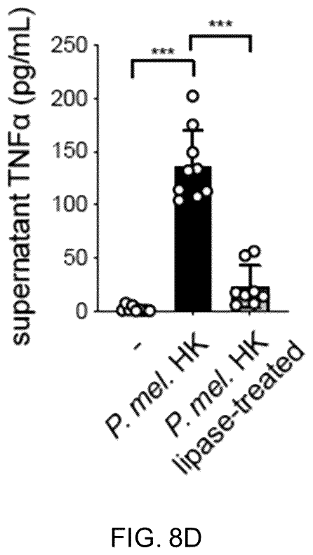

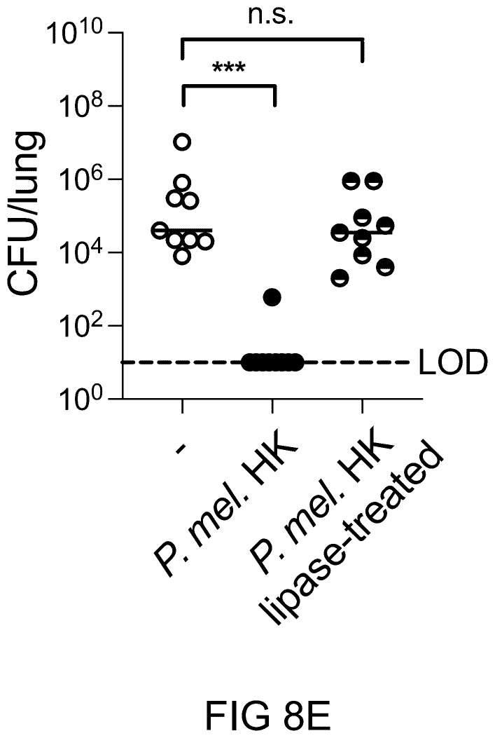

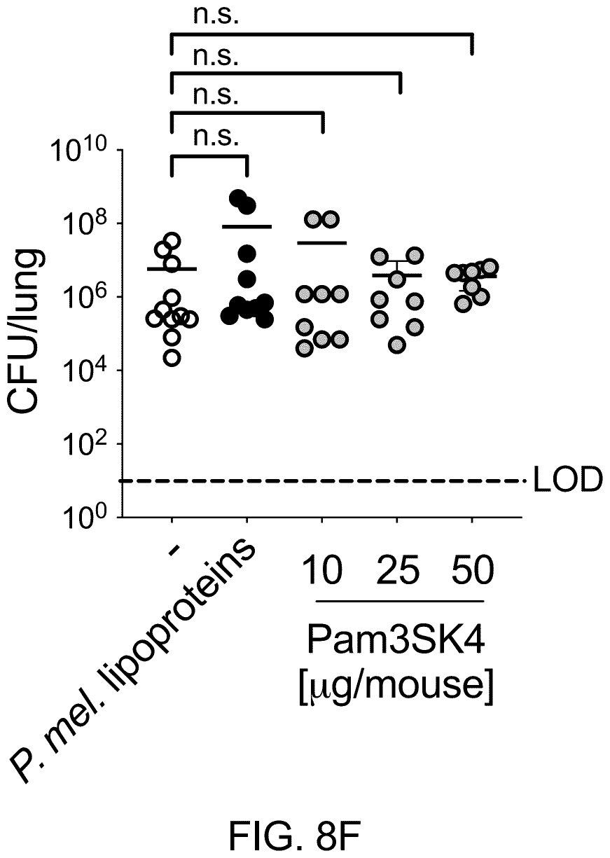

Results are shown in FIGS. 8A-8F, in which data are pooled from three independent experiments, with cells plated in triplicate for in vitro assays. Data are displayed as mean±SEM. Specifically, FIG. 8A shows supernatant TNFα detected 24 hours following incubation of bone marrow (BM) neutrophils purified from naïve wild-type (WT) mice with P. mel. strain 25845 HK at the indicated ratios (n=3 independent experiments/group). In neutrophils purified from the bone marrow of naïve mice, exposure to P. melaninogenica HK induced TNFα secretion in a dose-dependent manner (FIG. 8A). *p=0.0107, ***p<0.0001, one-way ANOVA with Dunnett's post hoc test.

Small molecule inhibitors of toll-like receptor (TLR) 2 (C29) and TLR4 (TAK-242) were used to compare the relative roles of TLR2 versus TLR4 for P. melaninogenica-induced TNFα. FIG. 8B shows supernatant TNFα detected 24 hours following incubation of BM neutrophils from WT mice incubated with P. mel. HK (1:1 ratio) with and without inhibitors of TLR2 (C29) or TLR4 (TAK-242), (n=3 independent experiments/group). Inhibition of TLR2, but not TLR4, resulted in loss of P. melaninogenica-induced neutrophil TNFα (FIG. 8B), suggesting that TLR2 signaling is involved in this response. From left to right ***p<0.0001, ***p<0.0001, p=0.8011, one-way ANOVA with Dunnett's post hoc test.

The results shown in FIG. 8C support those of FIG. 8B. Specifically, FIG. 8C shows supernatant TNFα detected 24 hours following incubation of BM neutrophils from WT or Tlr2−/− mice with PBS (-), P. mel. HK (1:1 ratio), P. mel. lipopolysaccharide (LPS, 10 ng/mL), P. mel. lipoproteins (10 ng/mL), or Pam3SK4 (10 ng/mL), (n=3 independent experiments/group). P. melaninogenica-induced TNFα secretion was absent in neutrophils purified from TLR2 deficient (Tlr2−/−) mice, in contrast to neutrophils from wild-type (WT) mice (FIG. 8C). These data indicate that TLR2 is involved in P. melaninogenica stimulation of neutrophil TNFα secretion. c From left to right, ***p<0.0001, p>0.9999, ***p<0.0001, ***p<0.0001, two-way ANOVA with Sidak's post hoc test.

Lipoproteins, a component of the cell membrane in both Gram-negative and Gram-positive bacteria, are a known bacterial TLR2 ligand. In contrast, TLR4 is responsive to LPS. Lipoproteins and LPS were purified from P. melaninogenica and the former, but not the latter, induced neutrophil TNFα secretion in a TLR2-dependent manner (FIG. 8C).

The digestion of bacterial lipoproteins with lipoprotein lipase is known to abrogate TLR2 activation. Lipoprotein lipase-treated P. melaninogenica HK was prepared as described above. Supernatant TNFα detected 24 hours following incubation of BM neutrophils from WT mice treated with PBS (-), untreated P. mel. HK, or lipoprotein-digested P. mel. HK (n=3 independent experiments/group) is shown in FIG. 8D. The results demonstrate that lipoprotein lipase digestion of P. melaninogenica resulted in significant loss of neutrophil TNFα secretion in vitro (FIG. 8D). ***p<0.0001, one-way ANOVA with Tukey's post hoc test.

Lipoprotein lipase-treated P. melaninogenica was also no longer protective against S. pneumoniae infection in mice (FIG. 8E). FIG. 8E shows lung type 2 S. pneumoniae burdens in mice treated with PBS (-), untreated P. mel. HK, or lipoprotein-digested P. mel. HK i.t. prior to 24 hour S. pneumoniae infection at 5×10⁶ CFU/mouse (n=9 mice/group). From left to right ***p=0.0004, p=0.1526, Kruskal-Wallis with Dunn's post hoc test.

However, as shown in FIG. 8F, lipoprotein-TLR2 signaling was not sufficient for protection, as neither the TLR2 agonist Pam3SK4 nor purified P. melaninogenica lipoproteins altered lung burdens of S. pneumoniae compared to mice treated with PBS. FIG. 8F shows lung S. pneumoniae burdens in mice treated with PBS (-, n=11 mice), P. mel. lipoproteins (10 μg/mouse, n=9 mice), or Pam3SK4 (10 μg/mouse, n=10 mice, 25 μg/mouse, n=8 mice, or 50 μg/mouse, n=8 mice) i.t. prior to a 24-hour S. pneumoniae infection. From left to right, p=0.9344, p=0.1490, p>0.9999, p>0.9999, Kruskal-Wallis with Dunn's post hoc test.

Together, the foregoing findings indicate that P. melaninogenica lipoproteins activate TLR2-dependent secretion of TNFα in neutrophils and appear necessary, but not sufficient, for P. melaninogenica-mediated protection against S. pneumoniae.

### Example 9: Role of TLR2 in P. melaninogenica-Mediated Protection (In Vivo)

The role of TLR2 in P. melaninogenica-induced immune activation and protection was further investigated in vivo as described in more detail below.

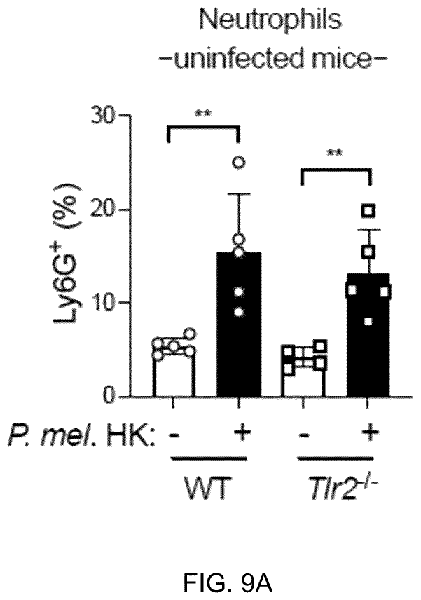

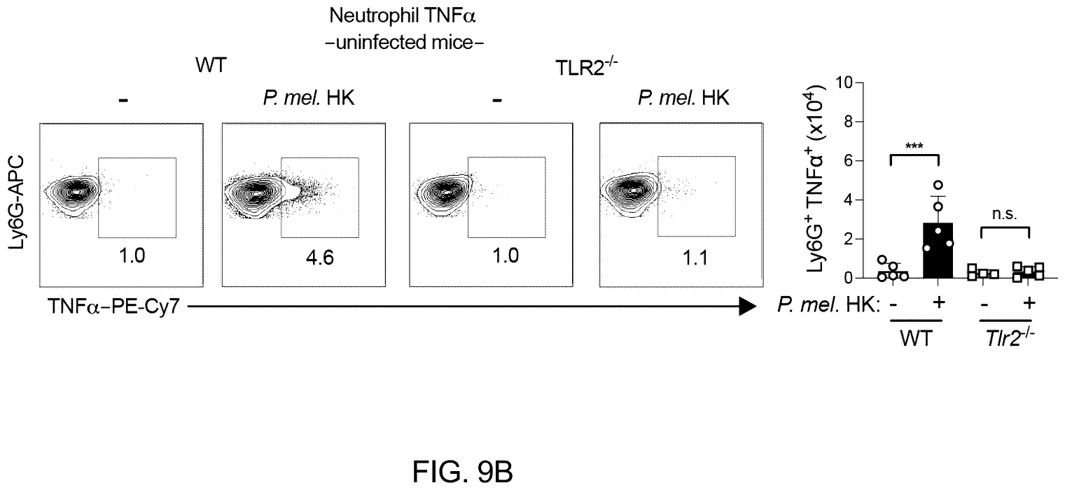

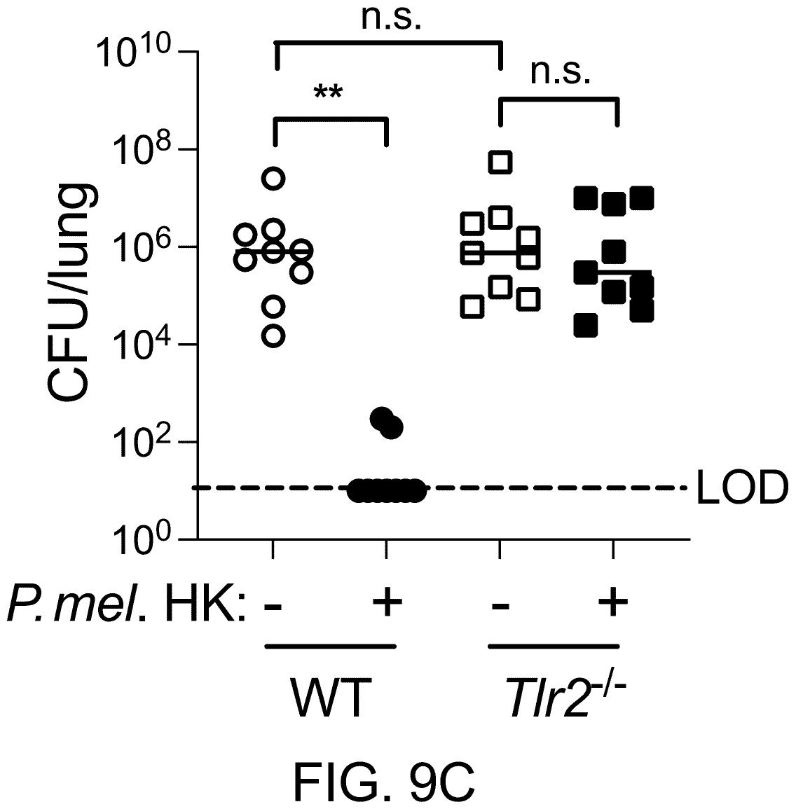

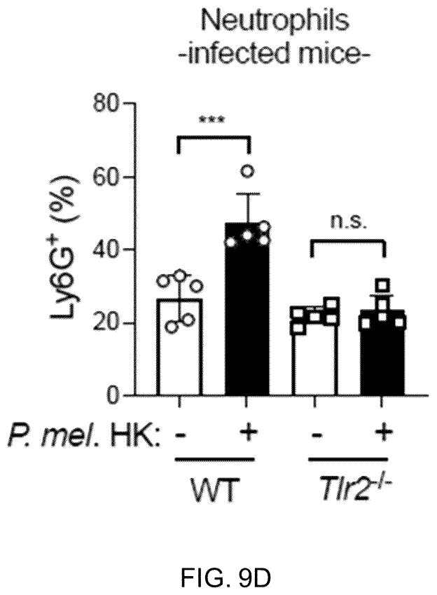

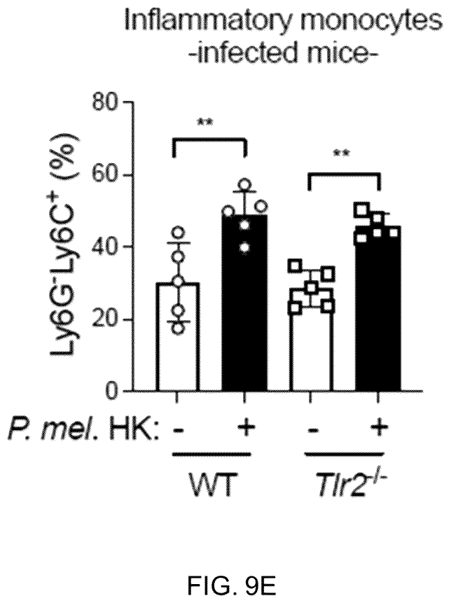

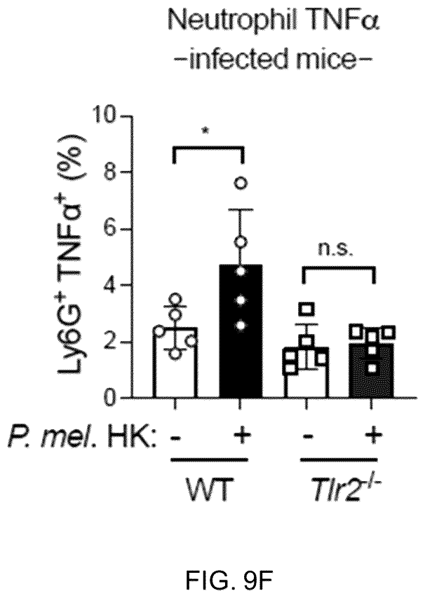

Results are shown in FIGS. 9A-9F, in which data are pooled from three independent experiments (FIG. 9C) or are representative from one of four independent experiments (FIGS. 9A, 9B, & 9D-9F). Data are displayed as mean±SEM. Specifically, FIGS. 9A and 9B show the percentage of neutrophils (9A) or representative flow cytometry plots and total cell numbers of neutrophil TNFα (9B) detected by intracellular flow cytometry in WT or Tlr2−/− mice treated with either PBS (-) or P. mel. strain 25845 heat-killed (HK) i.t. for 24 hours (n=5 mice/group). In Tlr2−/− mice exposed to P. melaninogenica HK alone (uninfected), the recruitment of both neutrophils and inflammatory monocytes was similar to that in WT mice (FIG. 9A), and CD11bhi DC and AM populations were unaffected (data not shown). However, P. melaninogenica-induced neutrophil TNFα was lost in Tlr2−/− mice (FIG. 9B). These findings indicate that TLR2 may be required for P. melaninogenica-induced neutrophil TNFα, but not P. melaninogenica-induced myeloid cell recruitment.

For FIG. 9A, **p=0.0026 (WT), **p=0.009 (Tlr2−/−), one-way ANOVA with Sidak's post hoc test, and p=0.0338 (WT), p=0.0081 (Tlr2−/−), Kruskal-Wallis with Dunn's post hoc test. For FIG. 9B, ***p=0.0002 (WT), p=0.9858 (Tlr2−/−), one-way ANOVA with Sidak's post hoc test, and p=0.0165 (WT), p>0.9999 (Tlr2−/−), Kruskal-Wallis with Dunn's post hoc test.

FIGS. 9C-9F show lung type 2 S. pneumoniae burdens (n=9 mice/group) (9C), percentage of neutrophils (n=5 mice/group) (9D), percentage of inflammatory monocytes (n=5 mice/group) (9E), and percentage of neutrophil TNFα (n=5 mice/group) (9F) detected by intracellular flow cytometry in mice treated with either PBS (-) or P. melaninogenica (P. mel.) HK i.t. prior to 24-hour S. pneumoniae infection at 5×10⁶ CFU/mouse.

In Tlr2−/− mice infected with S. pneumoniae, lung burdens were elevated regardless of Prevotella exposure, in contrast to WT mice in which Prevotella exposure was protective (FIG. 9C). P. melaninogenica-induced neutrophil recruitment was lost in Tlr2−/− mice infected with S. pneumoniae, while the recruitment of inflammatory monocytes was maintained (FIG. 9D & 9E). These data indicate that Prevotella-induced neutrophil recruitment is TLR2-dependent in S. pneumoniae-infected mice, a setting which may require TNFα feedback.

Neutrophils in the lungs of Tlr2−/− infected mice also no longer expressed TNFα in response to P. melaninogenica (FIG. 9F). The proportions of other lung myeloid cells, including CD11bhi DCs and AMs, were similar between WT and Tlr2−/− mice infected with S. pneumoniae (data not shown). Together, these data may indicate a critical role for TLR2 in P. melaninogenica-induced neutrophil activation and protection against S. pneumoniae. The separation between neutrophil expression of TNFα, which was TLR2-dependent, and the recruitment of neutrophils and inflammatory monocytes, which were TLR2-independent in P. melaninogenica-exposed mice, indicate that myeloid cell recruitment is by itself insufficient to improve protection against S. pneumoniae, which appears to require the activation of cells including neutrophils.

For FIGS. 9C-9F: In 9C, from left to right, **p=0.0017, p>0.9999, p>0.9999, Kruskal-Wallis with Dunn's post hoc test; 9D, *p=0.0494 (WT), p>0.9999 (Tlr2−/−), Kruskal-Wallis with Dunn's post hoc test; 9E, **p=0.0014 (WT), **p=0.0026 (Tlr2−/−), one-way ANOVA with Sidak's post hoc test, and p=0.0162 (WT), p=0.0400 (Tlr2−/−), Kruskal-Wallis with Dunn's post hoc test; 9F, *p=0.0142 (WT), p=0.9898 (Tlr2−/−), one-way ANOVA with Sidak's post hoc test, and p=0.2056 (WT), p>0.9999 (Tlr2−/−), Kruskal-Wallis with Dunn's post hoc test.

### Example 10: Potential Mechanism of P. melaninogenica-Enhanced Killing

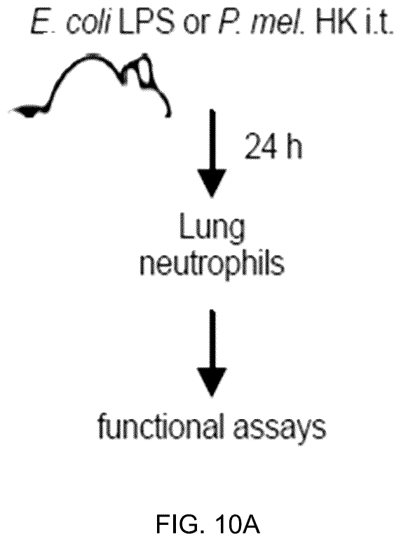

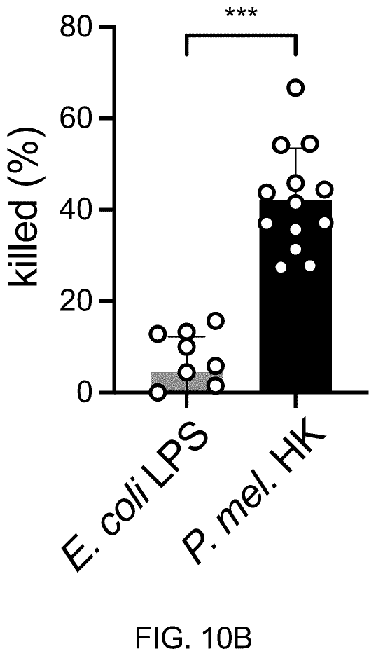

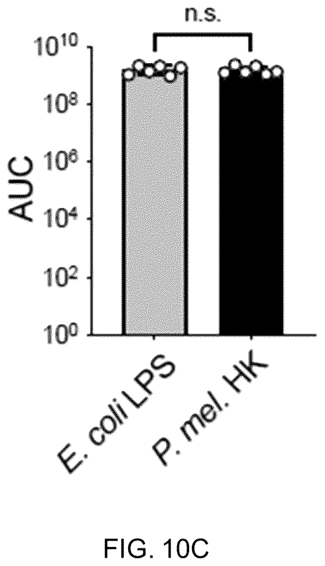

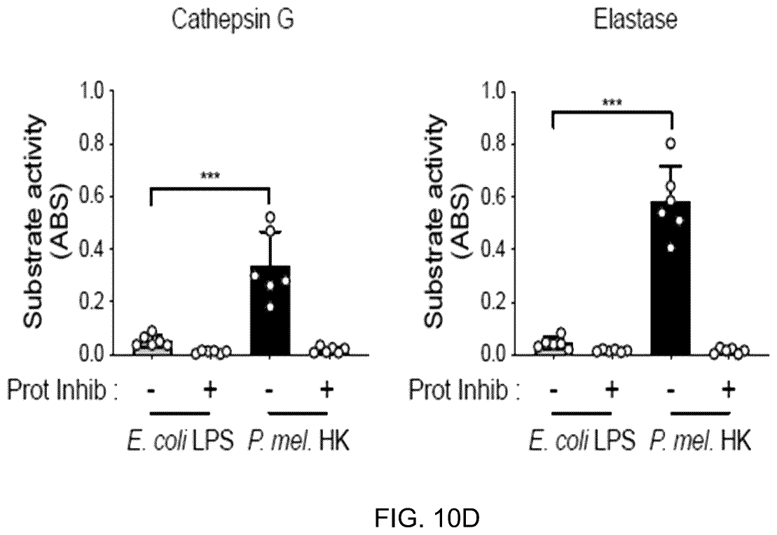

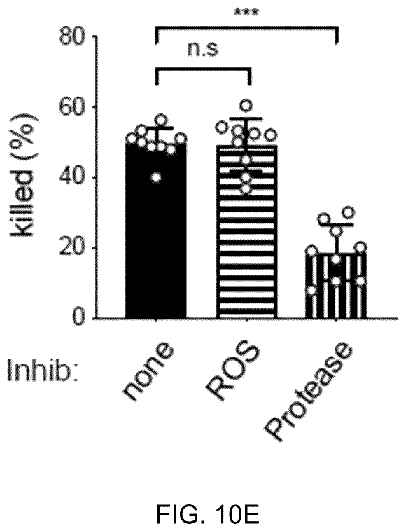

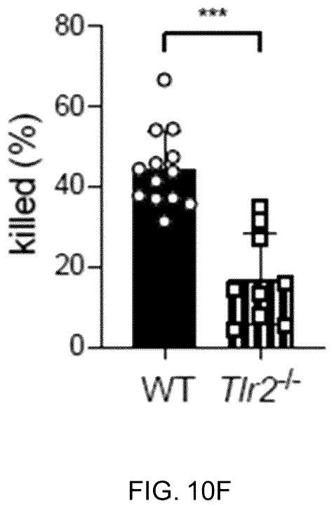

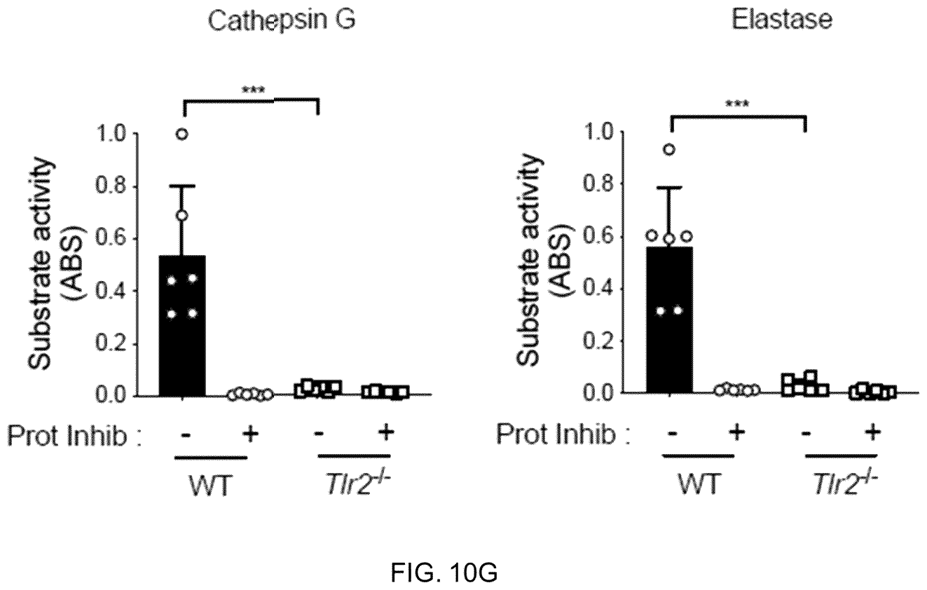

To address whether neutrophils activated by P. melaninogenica directly contribute to enhanced clearance of S. pneumoniae, neutrophil killing of S. pneumoniae was measured in vitro. Neutrophils purified from the bone marrow of naïve mice were no better at killing S. pneumoniae following pre-incubation with P. melaninogenica HK for up to 6 hours (data not shown), after which the viability of primary neutrophils declines. This suggests that while direct exposure to P. melaninogenica induces neutrophil secretion of TNFα, it is not sufficient to enhance killing of S. pneumoniae within this timeframe. Additional cells or signals in the lungs of P. melaninogenica-exposed mice might promote neutrophil killing of S. pneumoniae. To address this possibility, neutrophils were purified from the lungs of mice exposed to either P. melaninogenica or E. coli LPS for 24 hours (FIG. 10A). E. coli LPS was chosen as a non-protective immune stimulus that would still induce neutrophil recruitment. The neutrophils were used in functional assays as described in more detail below.

Results are shown in FIGS. 10B-10G, in which data are pooled from three independent experiments, with cells plated in duplicate or triplicate. Data are displayed as mean±SEM. Specifically, FIG. 10B shows the percent of type 2 S. pneumoniae killed by lung neutrophils purified from mice exposed to either E. coli lipopolysaccharide (LPS) or P. mel. strain 25845 heat-killed (HK) i.t. for 24 hours following a 1-hour incubation with S. pneumoniae opsonized with 3% fresh mouse serum (n=cells isolated from 11 mice/group). Neutrophils purified from the lungs of Prevotella-exposed mice killed significantly more S. pneumoniae than neutrophils purified from the lungs of mice exposed to E. coli LPS (FIG. 10B).

The two primary mechanisms for neutrophil killing of S. pneumoniae are the production of reactive oxygen species (ROS) and activity of serine proteases. Accordingly, ROS and serine proteases were investigated. FIG. 10C shows area under curve (AUC) for total ROS produced in 1 hour by lung neutrophils purified from mice exposed to either E. coli LPS or P. mel. HK i.t. for 24 hours, detected by luminol (n=cells isolated from 6 mice/group). FIG. 10D shows serine protease activity for cathepsin G and elastase+/− protease inhibitor cocktail (Prot Inhib) detected by substrate cleavage for lung neutrophils purified from WT mice exposed to either E. coli LPS or P. mel. HK (n=cells isolated from 6 mice/group). Neutrophils from P. melaninogenica-exposed and E. coli LPS-exposed mice produced similar levels of total ROS (FIG. 10C). In contrast, the activity of two serine proteases, cathepsin G and elastase, was significantly higher in neutrophils purified from the lungs of P. melaninogenica-exposed mice, compared to neutrophils from the lungs of mice exposed to E. coli LPS (FIG. 10D). Neutrophils pooled from naïve mice had similarly low serine protease activity as those from E. coli LPS-exposed mice (data not shown).

FIG. 10E shows the percent of S. pneumoniae killed by lung neutrophils purified from WT mice exposed to P. mel. HK i.t. for 24 hours following a 1-hour incubation with opsonized S. pneumoniae in the presence of the ROS inhibitor DPI (ROS), protease inhibitors (Protease), or no inhibitors (none) (n=cells isolated from 9 mice/group). The addition of protease inhibitors, but not the ROS inhibitor DPI, significantly reduced S. pneumoniae killing by neutrophils isolated from P. melaninogenica-exposed mice (FIG. 10E), without affecting S. pneumoniae growth (data not shown). Together, these data indicate that serine protease activity is involved in P. melaninogenica-enhanced killing in lung neutrophils.

The importance of TLR2 signaling for Prevotella-induced neutrophil killing was determined by comparing neutrophils purified from WT versus Tlr2−/− mice. Results are presented in FIGS. 10F and 10G, in which the percent of S. pneumoniae killed by lung neutrophils purified from WT or Tlr2−/− mice exposed to P. mel. HK i.t. for 24 hours (n=cells isolated from 13 WT mice, 9 Tlr2−/− mice) (10F) or serine protease activity +/− protease inhibitor cocktail (Prot Inhib) (n=cells isolated from 6 mice/group) (10G) is shown. Neutrophils from the lungs of Tlr2−/− mice exposed to P. melaninogenica were less efficient at killing S. pneumoniae compared to those from WT mice (FIG. 10F) and these cells had negligible serine protease activity (FIG. 10G), demonstrating a critical role for TLR2 in P. melaninogenica-enhanced neutrophil killing. Together, these findings suggest that P. melaninogenica exposure increases TLR2-dependent, serine protease-mediated killing of S. pneumoniae by lung neutrophils.

For FIGS. 10B-10G: In 10B, ***p<0.0001, two-tailed t-test; 10C, p=0.9566, two-tailed t-test; 10D, ***p=0.0004 (Cathepsin G), ***p<0.0001 (Elastase), two-tailed t-test; 10E, p=0.9926 (ROS), ***p<0.0001 (Protease), one-way ANOVA with Dunnett's post hoc test; 10F, ***p<0.0001, two-tailed t-test; and 10G, ***p=0.0008 (Cathepsin G), ***p=0.0002 (Elastase), two-tailed t-test.

### Example 11: Role of IL-10 in P. melaninogenica-Mediated Protection

Immune regulation is critical to mitigate the damaging effects of inflammation in the lung such as barrier disruption and reduced oxygen exchange. While TNFα primes several protective immune responses, overproduction causes tissue damage and impairs S. pneumoniae clearance. The anti-inflammatory cytokine IL-10 is a master regulator of pro-inflammatory responses including TNFα. In purified neutrophils, in addition to TNFα, P. melaninogenica HK induced the secretion of IL-10 in a dose-dependent and TLR2-dependent manner (data not shown). Also, neutrophil secretion of TNFα was inhibited in cultures exposed to both P. melaninogenica and S. pneumoniae, suggesting regulation of this response (data not shown). These findings mirror the systemic TNFα response in Prevotella-exposed mice, which was reduced following S. pneumoniae infection.

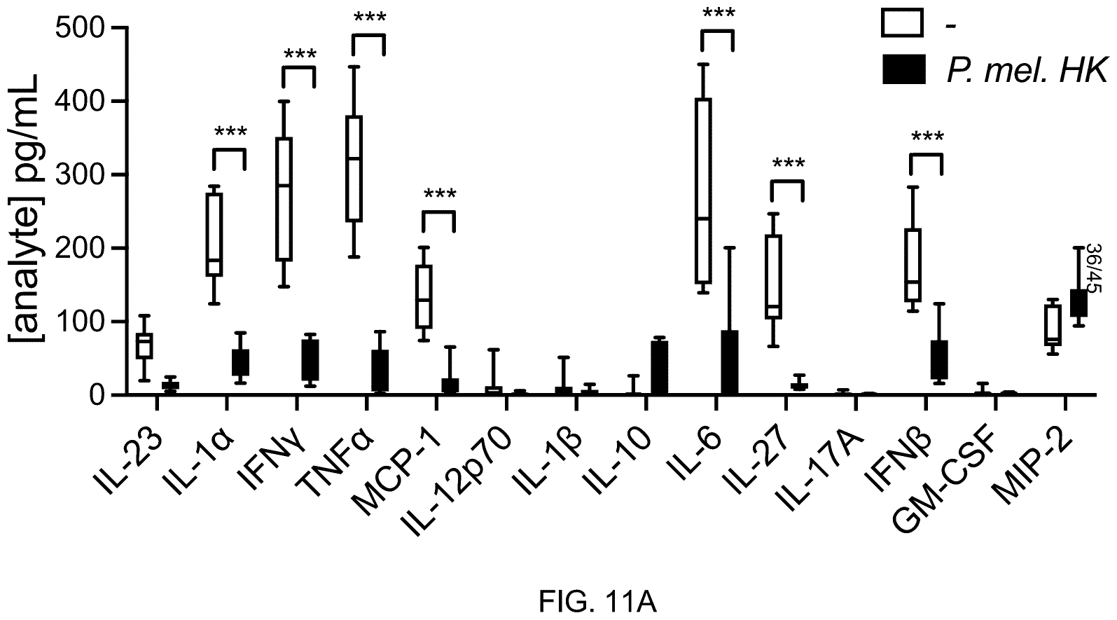

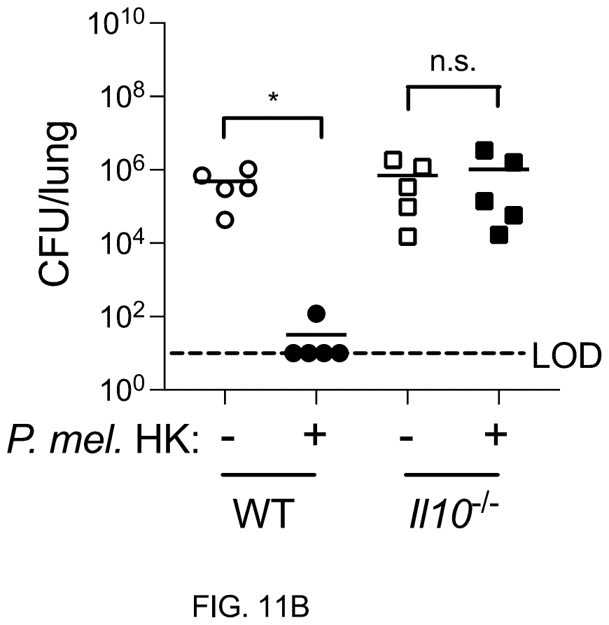

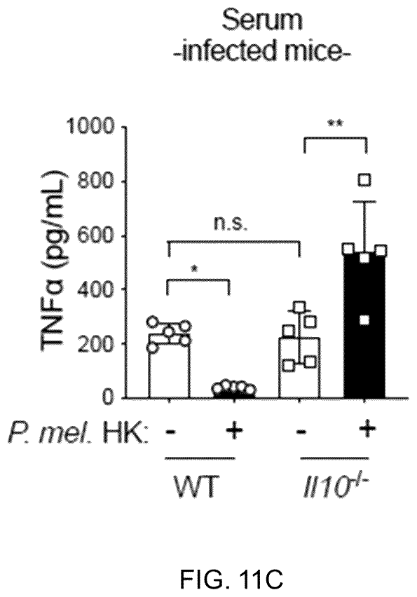

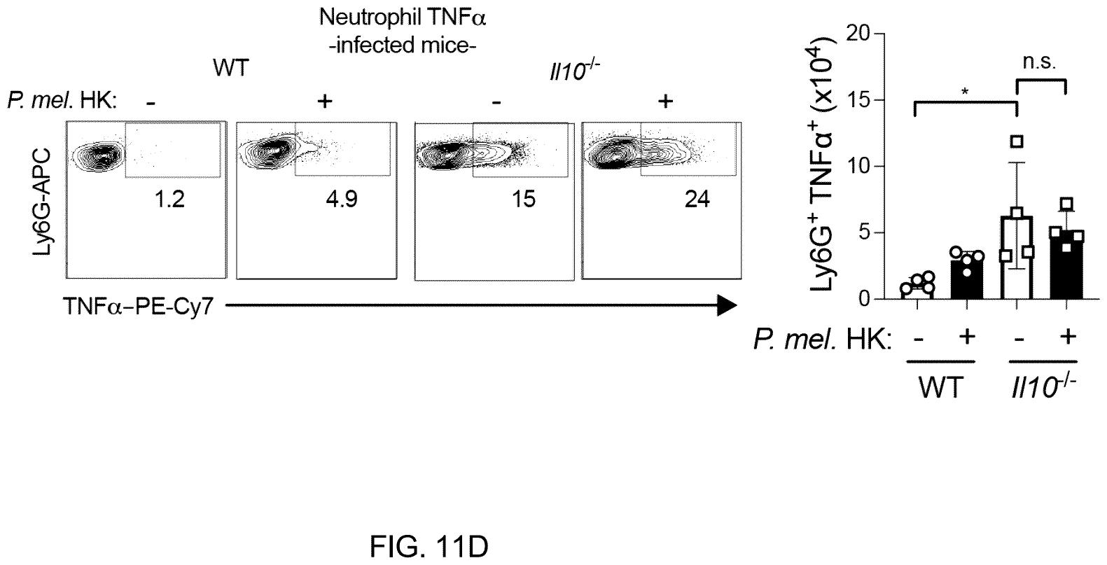

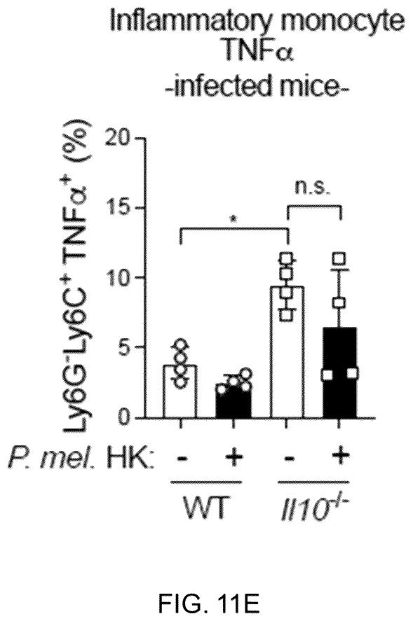

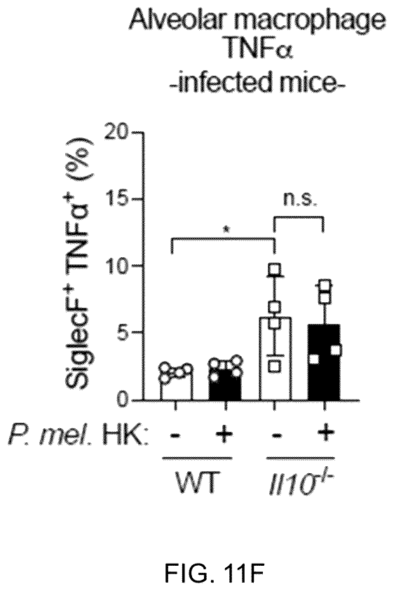

Cytokines and chemokines in lung bronchoalveolar lavage (BAL) were quantified following exposure to PBS (-) or P. mel. strain 25845 heat-killed (HK) i.t. prior to 24 h type 2 S. pneumoniae infection, 5×10⁶ CFU/mouse (n=9 mice/group). Results are shown in FIG. 11A. P. melaninogenica-exposed mice infected with S. pneumoniae had significantly reduced levels of several pro-inflammatory cytokines in lung BAL, including TNFα, IL-6, IL-1α, IFNβ, and IFNγ, compared to those infected with S. pneumoniae but not exposed to Prevotella. This is in contrast to uninfected mice, where P. melaninogenica exposure increased BAL pro-inflammatory cytokines at 24 hours, indicating that by 48 hours in co-infected mice, these responses were reduced. These data suggest that exposure to P. melaninogenica regulates S. pneumoniae-induced inflammation in the lung.

To address the potential role of regulatory IL-10 in P. melaninogenica-exposed mice, S. pneumoniae burdens in WT and IL-10 deficient (Il10−/−) mice with and without pre-exposure to P. melaninogenica HK were evaluated. FIGS. 11B and 11C show lung S. pneumoniae burdens (11B) and serum TNFα (11C) detected in WT or Il10−/− mice exposed to either PBS (-) or P. mel. HK i.t. prior to 24-hour S. pneumoniae infection (n=5 mice/group). Unexpectedly, P. melaninogenica-mediated protection against S. pneumoniae was lost in Il10−/− mice, which had similarly high burdens regardless of P. melaninogenica exposure (FIG. 11B). In Il10−/− mice, serum TNFα was elevated following P. melaninogenica exposure (FIG. 11C). In contrast, WT mice exposed to P. melaninogenica had reduced serum TNFα and elevated IL-10 (data not shown). These results indicate that P. melaninogenica-induced IL-10 regulates systemic TNFα during S. pneumoniae infection and is involved in P. melaninogenica-mediated protection.

Flow cytometry was used to evaluate TNFα in lung myeloid cells. FIGS. 11D-11F show representative flow cytometry plots and total cell numbers of neutrophil TNFα (11D), percentage of inflammatory monocyte TNFα (11E), and percentage of AM TNFα (11F) detected by intracellular flow cytometry from WT or Il10−/− mice treated with either PBS (-) or P. mel. HK i.t. prior to 24 h S. pneumoniae infection (n=4 mice/group). The results show that TNFα was significantly elevated in several cell types, including neutrophils, inflammatory monocytes, and AMs, in Il10−/− mice, regardless of P. melaninogenica exposure (FIGS. 11D-11F). Depletion of

TNFα was not sufficient to reverse the loss of P. melaninogenica-mediated protection in Il10−/− mice (data not shown), suggesting a broad loss of IL-10-mediated restraint of myeloid cell activation. Together, these findings reveal that IL-10 regulation of lung inflammation is a critical component of P. melaninogenica-mediated protection against S. pneumoniae infection. Overall, the results indicate that Prevotella induces sub-clinical inflammation associated with improved neutrophil killing of S. pneumoniae, which is followed by regulation of the inflammation by IL-10, which reduces infection-associated inflammation.

For FIGS. 11A-11F, data are pooled from three independent experiments (11A) or are representative from one of four independent experiments (11B-11F). Box boundaries in 11A indicate the 25th and 75th percentiles, with a horizontal line representing the median and whiskers indicating minimum and maximum values. Data in 11B-11F are displayed as mean±SEM. For 11A, ***p<0.0001, two-way ANOVA with Sidak's post hoc test; 11B, *p=0.0257 (WT), p>0.9999 (Il10−/−), Kruskal-Wallis with Dunn's post hoc test; 11C, from left to right, *p=0.0393, p=0.9973, **p=0.0012, one-way ANOVA with Tukey's post hoc test; 11D, from left to right, *p=0.0116, p=0.7388, one-way ANOVA with Sidak's post hoc test; 11E, from left to right, *p=0.0101, p=0.1698, one-way ANOVA with Sidak's post hoc test; and 11F, from left to right, *p=0.0280, p=0.9298, one-way ANOVA with Sidak's post hoc test.

### Example 12: Effects of Various Prevotella Strains

The effects of other airway Prevotella isolates were evaluated to determine if any, like P. melaninogenica, mediated a protective effect. All of Prevotella tannerae strain ATCCR 51259™ and Prevotella intermedia strain ATCCR 25611™ (American Type Culture Collection), Prevotella melaninogenica strain D18 (catalog #HM-80) and Prevotella buccae strain D17 (catalog #HM-80 and #HM-45, respectively; BEI resources, NIAID, NIH as part of the Human Microbiome project), and Prevotella nanceiensis strain PP1746 (gifted from Children's Hospital of Philadelphia) were grown as described in Example 1.

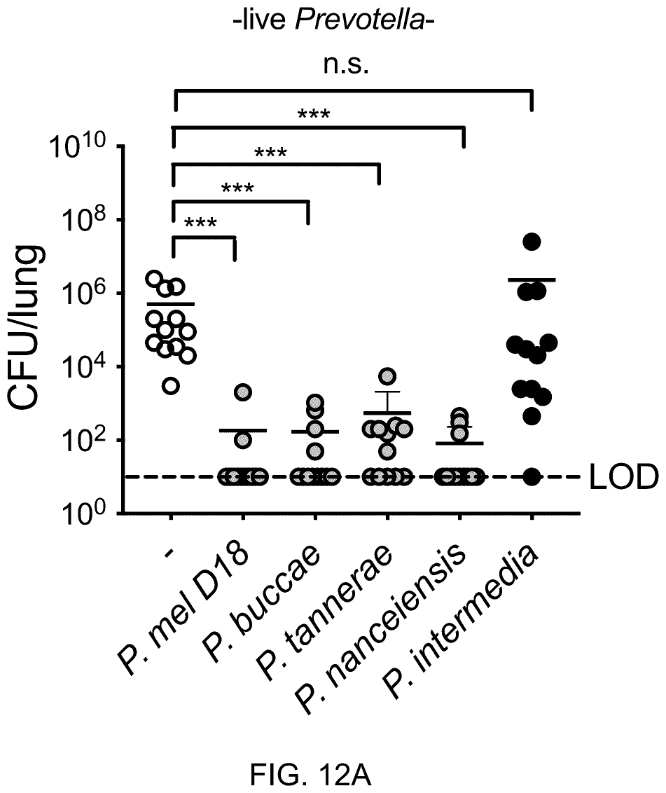

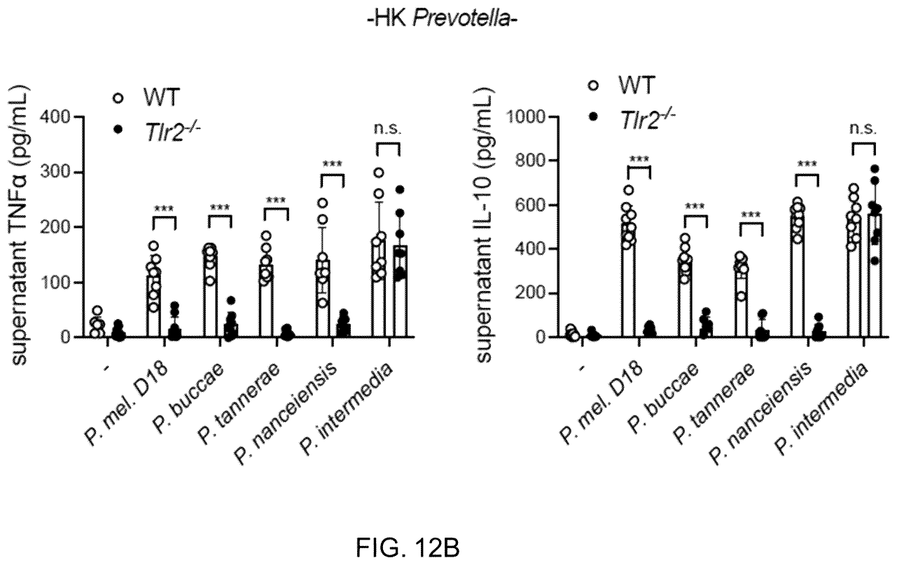

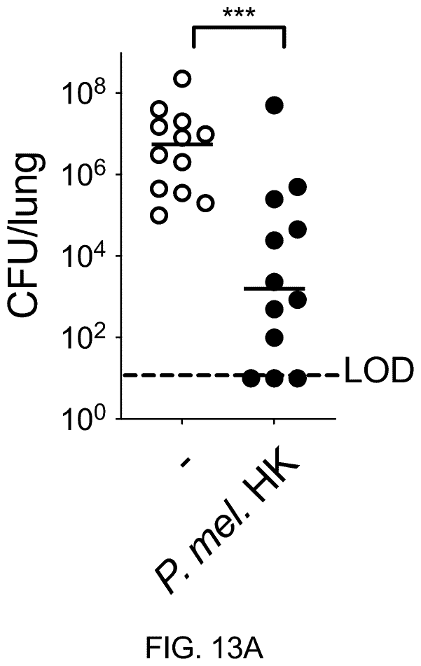

Results are shown in FIG. 12A, which depicts lung S. pneumoniae burdens in mice treated with PBS (-), live P. melaninogenica (P. mel.) strain D18, live P. buccae, live P. tannerae, live P. nanceiensis, or live P. intermedia i.t. prior to a 24-hour S. pneumoniae infection at 5×10⁶ CFU/mouse (n=12 mice/group). As with P. melaninogenica strain 25845, used throughout the foregoing Examples, exposure to live P. melaninogenica strain D18 significantly improved clearance of S. pneumoniae from the lung by 24 hours post-infection. Three additional live airway Prevotella species, including Prevotella buccae, Prevotella tannerae, and Prevotella nanceiensis, also increased S. pneumoniae clearance from the lung. In contrast, the periodontal pathogen P. intermedia was not protective. These data suggest that several airway Prevotella species are capable of enhancing protection against S. pneumoniae infection.

The ability of HK preparations of each Prevotella species to activate neutrophils purified from the bone marrow of WT versus Tlr2−/− mice was also investigated. Results are shown in FIG. 12B, which depicts supernatant cytokines TNFα and IL-10 detected 24 hours following incubation of BM neutrophils with P. mel. strain D18 heat-killed (HK), P. buccae HK, P. tannerae HK, P. nanceiensis HK, or P. intermedia HK (n=3 independent experiments/group). Similar to P. melaninogenica strain 25845, strain D18 as well as P. buccae, P. tannerae, and P. nanceiensis induced neutrophil secretion of TNFα and IL-10 in a TLR2-dependent manner. In contrast, P. intermedia activated neutrophils in a TLR2-independent manner. These data are consistent with a role for TLR2-dependent neutrophil activation in species of Prevotella that are protective against S. pneumoniae.

For FIGS. 12A & 12B, data are pooled from three independent experiments, with cells plated in triplicate for in vitro studies, displayed as mean±SEM. For FIG. 12A, from left to right, ***p<0.0001, ***p<0.0001, ***p=0.0005, ***p<0.0001, p=0.9999, Kruskal-Wallis with Dunn's post hoc test; and for FIG. 12B, from left to right, ***p<0.0001, ***p<0.0001, ***p<0.0001, ***p<0.0001, p=0.9959 (TNFα), ***p<0.0001, ***p<0.0001, ***p<0.0001, ***p<0.0001, p=0.9948 (IL-10), two-way ANOVA with Sidak's post hoc test.

### Example 13: Prevotella-Mediated Protection Against Staphylococcus aureus

The role of Prevotella in clearing another lung pathogen, S. aureus, was investigated. P. melaninogenica was grown and heat-killed as described in Examples 1 and 2. S. aureus strain USA300 was grown in broth from frozen stocks to mid-log phase and centrifuged at ≥20,000× g for 10 minutes followed by resuspension in PBS for infections. Inoculum burdens were determined by serial dilution for CFU enumeration. Lungs collected from infected mice were homogenized using a Bullet Blender tissue homogenizer (Stellar Scientific, Baltimore, MD). Tissue burdens were calculated following serial dilution in PBS and growth on Mannitol Salt agar plates. Plates were grown at 37° C. with 5% CO₂ for 24 hours. Mice were exposed to PBS (“-”), E. coli LPS, or P. melaninogenica HK i.t. as described above prior to infection with S. aureus at 10⁷ CFU/mouse. All infections were conducted in a volume of 50 μL on mice anesthetized by inhaled isoflurane. Neutrophils were isolated from the lungs, and opsonophagocytic killing was assayed, both as described above.

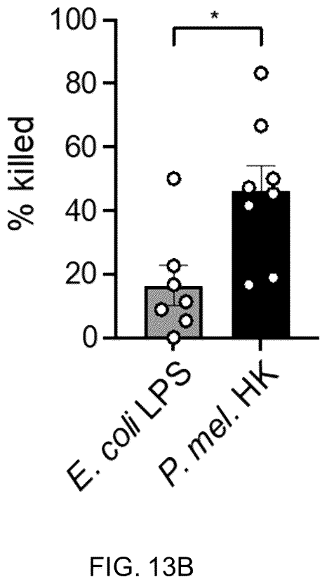

Results are shown in FIGS. 13A & 13B, in which data were pooled from 3 independent experiments with n=12 mice per group (13A) or n=6-7 mice/group (13B). FIG. 13A demonstrates that pre-exposure to inactivated (heat-killed) P. melaninogenica significantly (***p<0.001, Mann Whitney U test) improves clearance of the pathogen S. aureus from the lungs of mice. The enhanced clearance is associated with significantly (*p<0.05, t-test) improved neutrophil killing activity against S. aureus for neutrophils purified from the lungs of mice 24 hours following P. melaninogenica exposure (FIG. 13B).

Although various embodiments have been described above with a certain degree of particularity, or with reference to one or more individual embodiments, those skilled in the art could make numerous alterations to the disclosed embodiments without departing from the spirit or scope of this disclosure. Other embodiments are therefore contemplated. All matter contained in the above description and the accompanying drawings is illustrative only of particular embodiments and not limiting. Changes in detail, structure, or order of operation of steps of a method may be made without departing from the basic elements described herein.

What is claimed is:

1. A method of treating bacterial pneumonia, the method comprising administering a therapeutically effective amount of a composition comprising cells, or portions thereof, of one or more strains of Prevotella.

2. A method of promoting clearance of pneumonia-causing bacteria from a lung, the method comprising administering a composition comprising cells, or portions thereof, of one or more strains of Prevotella.

3. A method of reducing one or more symptoms caused by bacterial pneumonia, the method comprising administering a therapeutically effective amount of a composition comprising cells, or portions thereof, of one or more strains of Prevotella.

4. The method of any one of claims 1-3, wherein the one or more strains of Prevotella comprise P. melaninogenica, P. buccae, P. tannerae, or P. nanceiensis.

5. The method of any one of claims 1-4, wherein the administering is by inhalation.

6. The method of claim 2, wherein the pneumonia-causing bacteria are one or more of Streptococcus pneumoniae and Staphyloccocus aureus.

7. An inhalable immunobiotic composition comprising cells, or portions thereof, of one or more strains of Prevotella.

8. The immunobiotic composition of claim 7, wherein the one or more strains of Prevotella comprise P. melaninogenica, P. buccae, P. tannerae, or P. nanceiensis.

9. The immunobiotic composition of claim 7 or claim 8, wherein the portions of cells include lipoproteins.

10. The immunobiotic composition of claim 7 or claim 8, wherein the cells of at least one of the one or more strains of Prevotella are live.

11. The immunobiotic composition of claim 10, wherein the cells are present at about 10⁵ to about 10⁷ CFU.

12. The immunobiotic composition of claim 7 or claim 8, wherein the cells, or portions thereof, of at least one of the one or more strains of Prevotella are inactivated.

13. The immunobiotic composition of claim 12, wherein the cells, or portions thereof, are present at about 10⁷ CFU equivalents.

14. A method of promoting neutrophil activation in a lung, the method comprising administering a composition comprising cells, or portions thereof, of one or more strains of Prevotella.

15. The method of claim 14, wherein the one or more strains of Prevotella comprise P. melaninogenica, P. buccae, P. tannerae, or P. nanceiensis.

16. The method of claim 14 or claim 15, wherein the administering is by inhalation.

17. A method of activating, enhancing, and/or promoting an innate immune response in a subject afflicted with a respiratory infection, the method comprising administering a composition comprising cells, or portions thereof, of one or more strains of Prevotella.

18. The method of claim 17, wherein activating, enhancing, and/or promoting the innate immune response comprises increasing a presence of neutrophils in one or both lungs of the subject.

19. The method of claim 18, wherein increasing the presence of neutrophils causes a lung-localized increase in TNFα production followed by IL-10 production.

20. The method of claim 18 or claim 19, wherein activating, enhancing, and/or promoting the innate immune response comprises sub-clinical inflammation followed by inflammatory resolution.
